(19) United States
(12) Patent Application Publication
(10) Pub. No.: US 2009/0268031 A1
(43) Pub. Date: Oct. 29, 2009

(54) Electric Device, Information Terminal, Electric Refrigerator, Electric Vacuum Cleaner, Ultraviolet Sensor, and Field-Effect Transistor

(76) Inventors: Kazunari Honma, Gifu (JP); Mamoru Arimoto, Gifu (JP); Hitoshi Hirano, Hyogo (JP); Satoru Shimada, Gifu (JP)

Correspondence Address: MCDERMOTT WILL & EMERY LLP, 600 13TH STREET, N.W., WASHINGTON, DC, 20005-3096, US

(21) Appl. No.: 11/992,137

(22) PCT Filed: Sep. 13, 2006
(86) PCT No.: PCT/JP2006/318122

§ 371 Date: Jun. 23, 2009

(30) Foreign Application Priority Data
Sep. 15, 2005 (JP) 2005-267949
Oct. 28, 2005 (JP) 2005-313682
Nov. 30, 2005 (JP) 2005-344803
Dec. 20, 2005 (JP) 2005-365660
Jan. 26, 2006 (JP) 2006-017392

(51) Int. Cl. H04N 5/30; H01L 31/112

(52) U.S. Cl. 348/162; 257/290; 257/E31.079; 348/E05.085

(57) ABSTRACT

An electric device enabling the user to visually judge the section of present and amount of a substance absorbing or reflecting ultraviolet radiation. The electric device comprises an image detecting portion (6, 66, 127, 149) for receiving ultraviolet radiation and detecting an image from the received ultraviolet radiation and a display section (2, 32, 42, 52, 62, 82, 92, 102, 126, 147, 172) for displaying ultraviolet radiation information created from the image formed by the detected ultraviolet radiation by the image detecting portion.

### RELATED APPLICATIONS

This application is the U.S. National Phase under 35 U.S.C. § 371 of International Application No. PCT/JP2006/318122, filed on Sep. 13, 2006, which in turn claims the benefit of Japanese Application Nos. 2005-267949, 2005-313682, 2005-344803, 2005-365660, 2006-017392, filed on Sep. 15, 2005, Oct. 28, 2005, Nov. 30, 2005, Dec. 20, 2005, and Jan. 26, 2006, respectively, the disclosures of which Applications are incorporated by reference herein.

### TECHNICAL FIELD

The present invention relates to an electric device, an information terminal, an electric refrigerator and an electric vacuum cleaner, and more particularly, it relates to an electric device, an information terminal, an electric refrigerator and an electric vacuum cleaner having a display section.

### BACKGROUND ART

An information terminal capable of displaying an ultraviolet index based on the amount or intensity of ultraviolet radiation on a display section is known in general. Such an information terminal is disclosed in Japanese Patent Laying-Open No. 2004-23520, for example. The information terminal includes a cellular phone, a personal digital assistant, a laptop personal computer, a digital camera (electronic still camera) and the like. The information terminal disclosed in the aforementioned Japanese Patent Laying-Open No. 2004-23520 includes an ultraviolet radiation sensor for detecting the amount or intensity of ultraviolet radiation, and is formed such that the ultraviolet index based on the amount or intensity of ultraviolet radiation detected by the ultraviolet radiation sensor is displayed on the display section.

In the aforementioned Japanese Patent Laying-Open No. 2004-23520, however, the ultraviolet radiation sensor provided in the information terminal has only a function of detecting the amount or intensity of ultraviolet radiation in a region where the information terminal is located, and hence, in an object including a substance absorbing ultraviolet radiation, it is disadvantageously difficult to visually judge the section of presence and amount of the substance absorbing ultraviolet radiation in the object, for example.

### DISCLOSURE OF THE INVENTION

The present invention has been proposed in order to solve the aforementioned problem, and an object of the present invention is to provide an electric device, an information terminal, an electric refrigerator and an electric vacuum cleaner capable of visually determining the position or quantity of a substance absorbing or reflecting ultraviolet radiation.

In order to attain the aforementioned object, an electric device according to a first aspect of the present invention comprises an image detecting portion for receiving ultraviolet radiation and detecting an image by the received ultraviolet radiation, and a display section for displaying ultraviolet radiation information generated on the basis of the image by the ultraviolet radiation detected with the image detecting portion.

In the electric device according to the first aspect, as hereinabove described, the image detecting portion for receiving the ultraviolet radiation and detecting the image by the received ultraviolet radiation and the display section for displaying ultraviolet radiation information generated on the basis of the image by the ultraviolet radiation detected with the image detecting portion are provided, whereby the image by the received ultraviolet radiation can be detected with the image detecting portion and the image as ultraviolet radiation information generated on the basis of the image by the ultraviolet radiation detected with the image detecting portion can be displayed on the display section. Consequently, the image by the received ultraviolet radiation can be visually recognized with the electric device.

In the aforementioned electric device according to the first aspect, the image detecting portion preferably includes an ultraviolet radiation sensor having a substrate, first and second electrodes arranged at a prescribed interval along a surface of the substrate on the substrate, and a semiconductor layer capable of detecting the ultraviolet radiation, arranged on a portion between the first and second electrodes so as to be embedded. According to this structure, the first and second electrodes are arranged along the surface of the substrate and hence no electrode absorbing the ultraviolet radiation may be arranged on the light-receiving surface (upper surface) receiving the ultraviolet radiation of the semiconductor layer. Therefore, the semiconductor layer can directly receive the ultraviolet radiation. Thus, all the ultraviolet radiation incident from the light-receiving surface of the semiconductor layer can be received and hence the photosensitivity of the ultraviolet radiation can be increased. Consequently, clear image by the ultraviolet radiation can be detected.

In the aforementioned electric device according to the first aspect, the image detecting portion preferably includes a field-effect transistor having a semiconductor substrate, source and drain regions provided on the semiconductor substrate, a channel layer formed between the source and drain regions, a gate insulating film formed on the channel layer, and a gate electrode formed on the gate insulating film and formed with a light-receiving layer receiving the ultraviolet radiation to generate electrons and holes, a silicon oxide layer and an electrode layer in an order from a side closer to the gate insulating film. According to this structure, a current flowing between the source and drain regions changes according to the numbers of the electrons and holes generated due to the ultraviolet radiation incident upon the light-receiving layer when a prescribed constant voltage is applied between the source and drain regions, and hence the current flowing the source and drain regions is detected, whereby the ultraviolet radiation incident upon the light-receiving layer can be amplified and detected. Thus, the ultraviolet radiation can be detected with high photosensitivity. When a conductive material transparent with respect to the ultraviolet radiation is employed as the electrode layer, light is incident upon the light-receiving layer through the silicon oxide layer and the electrode layer transparent with respect to the ultraviolet radiation, whereby the ultraviolet radiation incident upon the light-receiving layer can be inhibited from being absorbed before reaching the light-receiving layer and hence reduction in the detection photosensitivity of the ultraviolet radiation can be suppressed. Consequently, the clear image by the ultraviolet radiation can be detected.

An information terminal according to a second aspect of the present invention comprises an image detecting portion for receiving ultraviolet radiation reflected on a surface of a prescribed object to thereby detect an image by the ultraviolet radiation reflecting the prescribed object, and a display section for displaying ultraviolet radiation information generated on the basis of the image by the ultraviolet radiation detected with the image detecting portion.

In this information terminal according to the second aspect, as hereinabove described, the image detecting portion for receiving ultraviolet radiation reflected on the surface of the prescribed object to thereby detect the image by the ultraviolet radiation reflecting the prescribed object and the display section for displaying ultraviolet radiation information generated on the basis of the image by the ultraviolet radiation detected with the image detecting portion are provided, whereby the image of the prescribed object by the ultraviolet radiation can be detected with the image detecting portion and the image as ultraviolet radiation information generated on the basis of the image by the ultraviolet radiation detected with the image detecting portion can be displayed on the display section. Consequently, the image of the prescribed object by the ultraviolet radiation can be visually recognized with the information terminal. The pigmented spot (black section) on the skin of the human body has a property of absorbing the ultraviolet radiation and hence the reflectance of the ultraviolet radiation on a section where the pigmented spot on the skin of the human body exists is smaller than that of the ultraviolet radiation on a section where no pigmented spot exists. When the image of the human body by the ultraviolet radiation is detected with the image detecting portion and the image by the ultraviolet radiation is displayed on the display section, the section where the pigmented spot on the skin of the human body exists and the section where no pigmented spot on the skin of the human body exists are different from each other in the detectable amount of the ultraviolet radiation with the image detecting portion, and hence the image of the human body by the ultraviolet radiation can be displayed on the display section such that the display color of the section where the pigmented spot on the skin of the human body exists and the display color of the section where no pigmented spot exists are different from each other. Therefore, the section where the pigmented spot on the skin of the human body exists can be confirmed with the information terminal. The antioxidant substances (polyphenol, flavone, flavonol, anthocyanin, lutein, chlorophyll and the like) contained in the vegetables and fruits each have a property of absorbing the ultraviolet radiation, and hence the reflectance of the ultraviolet radiation on the surface of the food containing the large quantity of antioxidant substances is smaller than that of the ultraviolet radiation on the surface of the food containing the small quantity of antioxidant substances. When the image of the food such as the vegetable or the fruit by the ultraviolet radiation is detected with the image detecting portion and the image by the ultraviolet radiation is displayed on the display section, the food containing a large quantity of antioxidant substances and the food containing a small quantity of antioxidant substances are different from each other in the detectable amount of the ultraviolet radiation with the image detecting portion, and hence the image of the food by the ultraviolet radiation can be displayed on the display section such that the food containing the large quantity of antioxidant substances and the food containing the small quantity of antioxidant substances are different from each other. Therefore, the food such as the vegetable or the fruit containing the large quantity of antioxidant substances and the food such as the vegetable or the fruit containing the small quantity of antioxidant substances can be distinguished from each other with the information terminal. It has been known that increase in the quantity of antioxidant substances (polyphenol, flavone, flavonol, anthocyanin, lutein, chlorophyll and the like) heightens the maturity of the food such as the vegetable or the fruit. In other words, the food such as the vegetable or the fruit containing the large quantity of antioxidant substances and the food such as the vegetable or the fruit containing the small quantity of antioxidant substances are distinguished from each other, whereby it is possible to distinguish the food such as the vegetable or the fruit whose maturity is high and the food such as the vegetable or the fruit whose maturity is low.

In the aforementioned information terminal according to the second aspect preferably further comprises an ultraviolet radiation filter through which the ultraviolet radiation is transmitted, wherein the ultraviolet radiation filter is arranged on a side closer to a light-receiving surface of the image detecting portion. According to this structure, only the ultraviolet radiation transmitting through the ultraviolet radiation filter is incident upon the light-receiving surface of the image detecting portion, and hence the image detecting portion can easily detect the image by the ultraviolet radiation. According to the structure in which the image detecting portion reacts against only the ultraviolet radiation, the ultraviolet radiation filter is not required.

The aforementioned information terminal according to the second aspect preferably further comprises a light-emitting portion emitting the ultraviolet radiation. According to this structure, the ultraviolet radiation is applied to the prescribed object by lighting the light-emitting portion, the image of the prescribed object by the ultraviolet radiation can be detected with the image detecting portion also under an environment where the amount of the ultraviolet radiation is small (in a room or at night, for example).

In the aforementioned information terminal according to the second aspect, the ultraviolet radiation information displayed on the display section may include at least an image generated on the basis of the image by the ultraviolet radiation detected with the image detecting portion, the prescribed object may be a human body including a skin having at least one of a black section absorbing the ultraviolet radiation and a section that is not black for a naked eye but absorbs the ultraviolet radiation, and an image capable of distinguishing at least one of the black section on the skin of the human body and the section that is not black for a naked eye but absorbs ultraviolet radiation may be displayed on the display section. According to this structure, the existence of the black section (pigmented spot) on the skin of the human body can be easily confirmed with the information terminal.

In the aforementioned information terminal according to the second aspect, the ultraviolet radiation information displayed on the display section may include at least an image generated on the basis of the image by the ultraviolet radiation detected with the image detecting portion, the prescribed object may be a food containing an antioxidant substance absorbing the ultraviolet radiation, and an image capable of distinguishing between a food containing a large quantity of antioxidant substances and a food containing a small quantity of antioxidant substances may be displayed on the display section. According to this structure, the food such as the vegetable or the fruit containing the large quantity of antioxidant substances (maturity is high) and the food such as the vegetable or the fruit containing the small quantity of antioxidant substances (maturity is low) can be easily distinguished from each other with the information terminal.

In this case, maturity of the food containing the antioxidant substance absorbing the ultraviolet radiation is preferably displayed on the display section in addition to the image by the ultraviolet radiation. According to this structure, the maturity of the food such as the vegetable or the fruit can be easily confirmed.

An electric refrigerator according to a third aspect of the present invention comprises a storage section storing an object, a light-emitting portion that applies ultraviolet radiation inside of the storage section, an image detecting portion for receiving the ultraviolet radiation reflected on a surface of an object stored in the storage section to thereby detect an image by the ultraviolet radiation reflecting the object stored in the storage section, and a display section for displaying ultraviolet radiation information generated on the basis of the image by the ultraviolet radiation detected with the image detecting portion.

In this electric refrigerator according to the third aspect, as hereinabove described, the image detecting portion for receiving the ultraviolet radiation reflected on the surface of an object stored in the storage section to thereby detect the image by the ultraviolet radiation reflecting the object stored in the storage section and the display section for displaying ultraviolet radiation information generated on the basis of the image by the ultraviolet radiation detected with the image detecting portion is provided, whereby the image of the object stored in the storage section by the ultraviolet radiation can be detected with the image detecting portion and the image as ultraviolet radiation information generated on the basis of the image by the ultraviolet radiation detected with the image detecting portion can be displayed on the display section. Consequently, the image of the object stored in the storage section by the ultraviolet radiation can be visually recognized without opening the electric refrigerator when the display section is mounted on the outside of the electric refrigerator. The antioxidant substances (polyphenol, flavone, flavonol, anthocyanin, lutein, chlorophyll and the like) contained in the food such as vegetable and fruit each have a property of absorbing the ultraviolet radiation, and hence the reflectance of the ultraviolet radiation on the surface of the food containing the large quantity of antioxidant substances is smaller than that of the ultraviolet radiation on the surface of the food containing the small quantity of antioxidant substances. When the image of the food such as the vegetable or the fruit by the ultraviolet radiation is detected with the image detecting portion and the image by the ultraviolet radiation is displayed on the display section, the food containing the large quantity of antioxidant substances and the food containing the small quantity of antioxidant substances are different from each other in the detectable amount of the ultraviolet radiation with the image detecting portion, and hence the image of the food by the ultraviolet radiation can be displayed on the display section such that the food containing the large quantity of antioxidant substances and the food containing the small quantity of antioxidant substances are different from each other. Therefore, the food such as the vegetable or the fruit containing the large quantity of antioxidant substances and the food such as the vegetable or the fruit containing the small quantity of antioxidant substances among the food such as the vegetable or the fruit stored in the storage section can be distinguished from each other. It has been known that increase in the quantity of the antioxidant substances (polyphenol, flavone, flavonol, anthocyanin, lutein, chlorophyll and the like) heightens the maturity of the food such as the vegetable or the fruit. In other words, the food such as the vegetable or the fruit containing the large quantity of antioxidant substances and the food such as the vegetable or the fruit containing the small quantity of antioxidant substances are distinguished from each other, whereby it is possible to distinguish the food such as the vegetable or the fruit whose maturity is high and the food such as the vegetable or the fruit whose maturity is low.

In the aforementioned electric refrigerator according to the third aspect, the ultraviolet radiation information displayed on the display section may include at least an image generated on the basis of the image by the ultraviolet radiation detected with the image detecting portion, the object stored in the storage section may include food each containing an antioxidant substance absorbing the ultraviolet radiation, and an image according to the quantity of antioxidant substances may be displayed on the display section. According to this structure, the food such as the vegetable or the fruit containing the large quantity of antioxidant substances (maturity is high) and the food such as the vegetable or the fruit containing the small quantity of antioxidant substances (maturity is low) among the foods such as the vegetables or the fruits stored in the storage section can be easily distinguished from each other.

In this case, maturity of the food containing the antioxidant substance absorbing the ultraviolet radiation is preferably displayed on the display section in addition to the image by the ultraviolet radiation. According to this structure, the maturity of the food such as the vegetable or the fruit stored in the storage section can be easily confirmed.

In the aforementioned structure in which the image according to the quantity of antioxidant substances is displayed on the display section, the electric refrigerator preferably further comprises a storage portion for storing the ultraviolet radiation information, wherein the ultraviolet radiation information displayed on the display section includes the ultraviolet radiation information stored in the storage portion in addition to the image by the ultraviolet radiation. According to this structure, the vegetable or the fruit in which the quantity of antioxidant substances increases (maturity is heightened) and the vegetable or the fruit in which the quantity of antioxidant substances decreases (maturity is lowered) can be distinguished from each other, and hence temporal change (temporal change of maturity) of the quantity of antioxidant substances of the same food can be confirmed. Thus, arbitrary peak ripeness of the food can be easily estimated. The arbitrary peak ripeness is the maturity of food arbitrarily selected by a person eating the food. When a person does not prefer the highest maturity (full maturity), for example, the peak ripeness that the person prefers can be determined since the state of a certain level of low maturity can be confirmed.

An electric vacuum cleaner according to the fourth aspect of the present invention comprises an image detecting portion for receiving ultraviolet radiation reflected on a surface of a prescribed region to thereby detect an image by the ultraviolet radiation reflecting the prescribed region, and a display section for displaying ultraviolet radiation information generated on the basis of the image by the ultraviolet radiation detected with the image detecting portion.

In this electric vacuum cleaner according to the fourth aspect, as hereinabove described, the image detecting portion for receiving ultraviolet radiation reflected on the surface of the prescribed region to thereby detect the image by the ultraviolet radiation reflecting the prescribed region, and the display section for displaying ultraviolet radiation information generated on the basis of the image by the ultraviolet radiation detected with the image detecting portion is provided, whereby the image of the prescribed region by the ultraviolet radiation can be detected with the image detecting portion and the image as ultraviolet radiation information generated on the basis of the image by the ultraviolet radiation detected with the image detecting portion can be displayed on the display section. Consequently, the image of the prescribed region by the ultraviolet radiation can be visually recognized with the electric device. The pollen has a property of absorbing the ultraviolet radiation having a wavelength of at most 400 nm and hence the reflectance of the ultraviolet radiation on a region where the pollen exists is smaller than that of the ultraviolet radiation on a region where no pollen exists. Thus, when the image of the prescribed region by the ultraviolet radiation is detected with the image detecting portion and the image by the ultraviolet radiation is displayed on the display section, the region where the pollen exists and the region where no pollen exists are different from each other in the detectable amount of the ultraviolet radiation with the image detecting portion, and hence the image of the prescribed region by the ultraviolet radiation can be displayed on the display section such that the display color of the region where the pollen on the prescribed region exists and the display color of the region where no pollen exists are different from each other. An insect or a bug shell thereof has a property of reflecting the ultraviolet radiation and hence the reflectance of the ultraviolet radiation on a region where the insect or the bug shell thereof exists is larger than that of the ultraviolet radiation on a region where no insect or no bug shell thereof exists. Thus, when the image of the prescribed region by the ultraviolet radiation is detected with the image detecting portion and the image by the ultraviolet radiation is displayed on the display section, the region where the insect or the bug shell thereof on the prescribed region exists and the region where no insect or no bug shell thereof exists are different from each other in the detectable amount of the ultraviolet radiation with the image detecting portion, and hence the image of the prescribed region by the ultraviolet radiation can be displayed on the display section such that the display color of the region where the insect or the bug shell thereof on the prescribed region exists and the display color of the region where no the insect or the bug shell thereof exists are different from each other. Therefore, the region where the pollen on the prescribed region exists and the region where the insect or the bug shell thereof exists can be confirmed with the electric vacuum cleaner. The insect is a microorganism having a property of reflecting the ultraviolet radiation, existing on a floor of a house, a flooring material, a carpet and a bedding such as a spider or a tick, for example.

The aforementioned electric vacuum cleaner according to the fourth aspect preferably further comprises an ultraviolet radiation filter through which the ultraviolet radiation is transmitted, wherein the ultraviolet radiation filter is arranged on a side closer to a light-receiving surface of the image detecting portion. According to this structure, only the ultraviolet radiation transmitting through the ultraviolet radiation filter is incident upon the light-receiving surface of the image detecting portion, and hence the image detecting portion can easily detect the image by the ultraviolet radiation. According to the structure in which the image detecting portion reacts against only the ultraviolet radiation, the ultraviolet radiation filter is not required.

The aforementioned electric vacuum cleaner according to the fourth aspect preferably further comprises a light-emitting portion emitting the ultraviolet radiation. According to this structure, the ultraviolet radiation is applied to the prescribed object by lighting the light-emitting portion, whereby the image of the prescribed region by the ultraviolet radiation can be detected with the image detecting portion also under an environment where the amount of the ultraviolet radiation is small (in a room or at night, for example).

In aforementioned electric vacuum cleaner according to the fourth aspect, the ultraviolet radiation information displayed on the display section may include at least an image generated on the basis of the image by the ultraviolet radiation detected with the image detecting portion, the prescribed region may be a cleaned region including a region where a pollen absorbing the ultraviolet radiation exists, and an image capable of distinguishing the pollen existing on the cleaned region may be displayed on the display section. According to this structure, the region where the pollen on the cleaned region exists can be easily confirmed with the electric vacuum cleaner.

In the aforementioned electric vacuum cleaner according to the fourth aspect, the ultraviolet radiation information displayed on the display section may include at least an image generated on the basis of the image by the ultraviolet radiation detected with the image detecting portion, the prescribed region may be a cleaned region including a region where an insect or a bug shell thereof reflecting the ultraviolet radiation exists, and an image capable of distinguishing the insect or the bug shell thereof existing on the cleaned region may be displayed on the display section. According to this structure, the region where the insect or the bag shell thereof exists on the cleaned region can be easily confirmed with the electric vacuum cleaner.

The aforementioned electric vacuum cleaner according to the fourth aspect preferably further comprises a first annunciation portion aurally announcing or a second annunciation portion visually announcing the ultraviolet radiation information generated on the basis of the image by the ultraviolet radiation detected with the image detecting portion. According to this structure, in a case where existence of the pollen, the insect or the bug shell thereof is announced when the region where the pollen, the insect or the bug shell exists is detected on the cleaned region with the first annunciation portion or the second annunciation portion, the operator can simply confirm the display section in the announcement and does not need to always monitor the display section.

An ultraviolet radiation sensor according to a fifth aspect of the present invention comprises a substrate, a first electrode and a second electrode arranged at a prescribed interval along a surface of the substrate on the substrate, and a semiconductor layer capable of detecting ultraviolet radiation, arranged on a portion between the first and second electrodes so as to be embedded.

In this ultraviolet radiation sensor according to the fifth aspect, as hereinabove described, the first electrode and the second electrode arranged at the prescribed interval along the surface of the substrate on the substrate and the semiconductor layer capable of detecting ultraviolet radiation, arranged on the portion between the first and second electrodes so as to be embedded are provided, whereby the first and second electrodes are arranged along the surface of the substrate and hence no electrode absorbing the ultraviolet radiation may be arranged on the light-receiving surface (upper surface) receiving the ultraviolet radiation of the semiconductor layer. Therefore, the semiconductor layer can directly receive the ultraviolet radiation. Consequently, all the ultraviolet radiation incident from the light-receiving surface of the semiconductor layer can be received and hence the photosensitivity of the ultraviolet radiation can be increased.

In the aforementioned ultraviolet radiation sensor according to the fifth aspect, the semiconductor layer preferably includes a silicon nanoparticle layer made of silicon nanoparticles. According to this structure, when the silicon nanoparticles of the silicon nanoparticle layer receive the ultraviolet radiation, the silicon nanoparticles obtain energy of the ultraviolet radiation and electrons and holes are excited, and hence the ultraviolet radiation sensor detecting only the ultraviolet radiation can be easily formed.

In the aforementioned structure comprising the silicon nanoparticle layer, the silicon nanoparticles of the silicon nanoparticle layer preferably each have a particle size capable of having a band gap of at least 3.1 eV. Such a silicon nanoparticle layer formed by the silicon nanoparticles is employed, whereby electrons can be excited from the silicon nanoparticles with the ultraviolet radiation having a wavelength of at most 400 nm (energy of at least 3.1 eV) while inhibiting electrons from being excited from the silicon nanoparticles with the visible light having a wavelength longer than 400 nm (energy of less than 3.1 eV). Consequently, electrons can be excited from the silicon nanoparticles over a band gap of at least 3.1 eV only when receiving the ultraviolet radiation having the wavelength of at most 400 nm, and hence the ultraviolet radiation sensor detecting only the ultraviolet radiation can be easily formed.

In the aforementioned ultraviolet radiation sensor according to the fifth aspect, the first electrode is preferably formed by a p-type semiconductor layer and the second electrode is preferably formed by an n-type semiconductor layer. According to this structure, electrons are required to be excited to the energy level from the valence band of the p-type semiconductor to the conduction band of the silicon nanoparticles of the silicon nanoparticle layer in order to excite electrons taking a role as a current from the p-type semiconductor where the quantity of electrons are small on a conduction band. Therefore, the energy on the band gap of the p-type semiconductor layer and the energy up to the energy level on the conduction band of the silicon nanoparticles are required to be provided to the electrons on the valence band of the p-type semiconductor in order to excite the electrons on the valence band of the p-type semiconductor to the energy level of the conduction band of the silicon nanoparticles of the silicon nanoparticle layer. When the visible light having a wavelength longer (energy smaller) than that of the ultraviolet radiation is incident, electrons can be inhibited from being excited from the p-type semiconductor layer. The electrons excited on the p-type semiconductor layer are likely to be bonded with holes, and hence is unlikely to contribute to a current. Thus, the electrons excited by the visible light can be inhibited from being detected as a current. Consequently, only holes and electrons excited by the ultraviolet radiation can be detected as a current, and hence detection accuracy of the ultraviolet radiation can be improved. In a structure where two n-type polysilicon layers are employed as electrodes, on the other hand, electrons are simply excited up to the energy level of the conduction band of the silicon nanoparticles of the silicon nanoparticle layers from the conduction band of the n-type semiconductor layer in order to excite electrons taking a role as a current from the n-type semiconductor layer where the quantity of electrons are large on the conductive band. In this case, only the energy up to the energy level of the conduction band of the silicon nanoparticles is simply provided to the electrons on the conduction band of the n-type semiconductor layer in order to excite the electrons on the conduction band of the n-type semiconductor layer up to the energy level of the conduction band of the silicon nanoparticles of the silicon nanoparticle layers. Thus, electrons are disadvantageously easily excited by small energy provided by the visible light when the visible light having a wavelength longer (energy smaller) than the ultraviolet radiation is incident upon the n-type semiconductor layer where the quantity of electrons is large. Therefore, the electrode are preferably formed by the p-type semiconductor layer and the n-type semiconductor layer as compared with the electrode formed by the two n-type semiconductor layers.

In the aforementioned ultraviolet radiation sensor comprising the first electrode formed by the p-type semiconductor layer and the second electrode formed by the n-type semiconductor layer, a first voltage is preferably applied to the first electrode formed by the p-type semiconductor layer, and a second voltage larger than the first voltage is preferably applied to the second electrode formed by the n-type semiconductor layer. According to this structure, electrons excited from the silicon nanoparticles of the silicon nanoparticle layer can be gravitated to a side of the n-type semiconductor layer from a side of the p-type semiconductor layer. Thus, the electrons excited from the silicon nanoparticles are detected as a current flowing between the p-type semiconductor layer and the n-type semiconductor layer, whereby the amount of the ultraviolet radiation can easily be detected. As hereinabove described, in the p-type semiconductor layer, electrons can be inhibited from being excited due to small energy of the received visible light, and hence electrons excited by the visible light can be inhibited from being detected as a current due to gravitation to a side of the n-type semiconductor of a high potential side.

In the aforementioned ultraviolet radiation sensor according to the fifth aspect, the first electrode and the second electrode preferably include a plurality of electrode sections respectively, and the plurality of electrode sections of the first electrode and the plurality of electrode sections of the second electrode are preferably arranged so as to be opposed to each other at prescribed intervals. According to this structure, a plurality of regions between the electrode sections of the first electrode and the electrode sections of the second electrode can be formed, and hence the area of receiving the ultraviolet radiation of silicon nanoparticle layer arranged on a plurality of regions formed is increased. Consequently, the amount of the ultraviolet radiation received by the silicon nanoparticle layer can be increased, and hence photosensitivity of the ultraviolet radiation can be further improved.

In this case, the first electrode and the second electrode are preferably formed integrally in comb-shapes including the plurality of electrode sections respectively. According to this structure, each electrode for applying a voltage is simply formed per one location with respect to the plurality of electrode sections of the first electrode and the plurality of electrode sections of the second electrode, and hence the structure can be simplified.

In the aforementioned ultraviolet radiation sensor according to the fifth aspect, the substrate preferably includes a conductive substrate, and the ultraviolet radiation sensor further comprises an insulating layer formed between the conductive substrate and the first and the second electrodes. According to this structure, electrical connection between the first and second electrodes and the conductive substrate can be suppressed by the insulating layer between the first and second electrodes also when the first and second electrodes are formed on the upper side of the conductive substrate. Consequently, a voltage is applied between the first and second electrodes, whereby the holes and the electrons excited from the silicon nanoparticles of the silicon nanoparticle layer can be easily detected as a current flowing between the first and second electrodes.

A field-effect transistor according to a sixth aspect of the present invention comprises a semiconductor substrate, a source region and a drain region provided on the semiconductor substrate, a channel layer formed between the source and drain regions, and a gate insulating film formed on the channel layer and a gate electrode formed on the gate insulating film, wherein the gate electrode includes a light-receiving layer receiving ultraviolet radiation to generate electrons and holes, a silicon oxide layer and an electrode layer in an order from a side closer to the gate insulating film.

As hereinabove described, the field-effect transistor according to the sixth aspect is formed such that the gate electrode includes the light-receiving layer receiving ultraviolet radiation to generate electrons and holes, the silicon oxide layer and the electrode layer in an order from the side closer to the gate insulating film, whereby a current flowing between the source and drain regions changes according to the numbers of the electrons and holes generated due to the ultraviolet radiation incident upon the light-receiving layer when a prescribed constant voltage is applied between the source and drain regions, and hence the current flowing the source and drain regions is detected, whereby the ultraviolet radiation incident upon the light-receiving layer can be amplified and detected. Thus, the ultraviolet radiation can be detected with high photosensitivity. When a conductive transparent material with respect to the ultraviolet radiation is employed as the electrode layer, light is incident upon the light-receiving layer through the silicon oxide layer and the electrode layer transparent with respect to the ultraviolet radiation, whereby the ultraviolet radiation incident upon the light-receiving layer can be inhibited from being absorbed before reaching the light-receiving layer and hence reduction in the detection photosensitivity of the ultraviolet radiation can be suppressed.

In the aforementioned field-effect transistor according to the sixth aspect, a particle size of each silicon nanoparticle of the light-receiving layer is preferably at least 0.4 nm and not more than 2 nm. According to this structure, the band gap of the light-receiving layer becomes at least 3.0 eV, whereby electrons are not excited from a valence band to a conduction band with visible light having a wavelength longer than 400 nm and electrons are selectively excited with ultraviolet radiation having a wavelength of at most 400 mm, and hence it is possible to provide the field-effect transistor more effectively detecting the ultraviolet radiation.

### BRIEF DESCRIPTION OF THE DRAWINGS

FIG. 1 A plan view showing a structure of a cellular phone (information terminal) according to a first embodiment of the present invention.

FIG. 2 A sectional view taken along the line 1000-1000 in FIG. 1.

FIG. 3 A sectional view taken along the line 1100-1100 in FIG. 1.

FIG. 4 A block diagram showing an inner structure of the cellular phone according to the first embodiment shown in FIG. 1.

FIG. 5 A plan view showing a structure of a personal digital assistant (information terminal) according to a first modification of the first embodiment of the present invention.

FIG. 6 A plan view showing a structure of a laptop personal computer (information terminal) according to a second modification of the first embodiment of the present invention.

FIG. 7 A plan view showing a structure of a digital camera (information terminal) according to a third modification of the first embodiment of the present invention.

FIG. 8 A plan view showing a structure of a cellular phone (information terminal) according to a second embodiment of the present invention.

FIG. 9 A sectional view taken along the line 2000-2000 in FIG. 8.

FIG. 10 A sectional view taken along the line 2100-2100 in FIG. 8.

FIG. 11 A block diagram showing an inner structure of the cellular phone according to the second embodiment shown in FIG. 8.

FIG. 12 A plan view showing a structure of a personal digital assistant (information terminal) according to a first modification of the second embodiment of the present invention.

FIG. 13 A plan view showing a structure of a laptop personal computer (information terminal) according to a second modification of the second embodiment of the present invention.

FIG. 14 A plan view showing a structure of a digital camera (information terminal) according to a third modification of the second embodiment of the present invention.

FIG. 15 A perspective view showing a structure of an electric refrigerator according to a third embodiment of the present invention.

FIG. 16 A plan view showing a protruding section of the electric refrigerator according to the third embodiment shown in FIG. 15.

FIG. 17 A sectional view taken along the line 3000-3000 in FIG. 16.

FIG. 18 A sectional view taken along the line 3100-3100 in FIG. 16.

FIG. 19 A block diagram showing an inner structure of the electric refrigerator according to the third embodiment shown in FIG. 15.

FIG. 20 A block diagram showing an inner structure of an electric refrigerator according to a modification of the third embodiment of the present invention.

FIG. 21 A perspective view showing a structure of an electric vacuum cleaner according to a fourth embodiment of the present invention.

FIG. 22 An enlarged view showing the vicinity of an opening of the electric vacuum cleaner according to the fourth embodiment shown in FIG. 21.

FIG. 23 A sectional view taken along the line 4000-4000 in FIG. 22.

FIG. 24 A sectional view taken along the line 4100-4100 in FIG. 22.

FIG. 25 A graph showing the relation between reflectance and wavelength of light of a flooring material, a carpet, a pollen and an insect or a bug shell.

FIG. 26 A block diagram showing an inner structure of the electric vacuum cleaner according to the fourth embodiment shown in FIG. 21.

FIG. 27 A perspective view showing a structure of an electric vacuum cleaner according to a modification of the fourth embodiment of the present invention.

FIG. 28 A block diagram showing an inner structure of the electric vacuum cleaner according to the modification of the fourth embodiment shown in FIG. 27.

FIG. 29 A plan view of an ultraviolet radiation sensor according to a fifth embodiment of the present invention.

FIG. 30 A sectional view taken along the line 5000-5000 in FIG. 29.

FIG. 31 A plan view of the ultraviolet radiation sensor from which an insulating layer and an electrode, according to the fifth embodiment shown in FIG. 29.

FIG. 32 A sectional view taken along the line 5100-5100 in FIG. 29.

FIG. 33 A sectional view taken along the line 5200-5200 in FIG. 29.

FIG. 34 A graph showing energy of light with respect to the wavelength of light.

FIG. 35 A band gap diagram of an n-type polysilicon layer, a p-type polysilicon layer and a silicon nanoparticle layer of the ultraviolet radiation sensor according to the fifth embodiment shown in FIG. 29.

FIG. 36 A band gap diagram of an n-type polysilicon layer and a silicon nanoparticle layer according to a comparative example of the ultraviolet radiation sensor according to the fifth embodiment shown in FIG. 29.

FIG. 37 A sectional view for illustrating a process of fabricating the ultraviolet radiation sensor according to the fifth embodiment shown in FIG. 29.

FIG. 38 A sectional view for illustrating a process of fabricating the ultraviolet radiation sensor according to the fifth embodiment shown in FIG. 29.

FIG. 39 A sectional view for illustrating a process of fabricating the ultraviolet radiation sensor according to the fifth embodiment shown in FIG. 29.

FIG. 40 A sectional view for illustrating a process of fabricating the ultraviolet radiation sensor according to the fifth embodiment shown in FIG. 29.

FIG. 41 A sectional view for illustrating a process of fabricating the ultraviolet radiation sensor according to the fifth embodiment shown in FIG. 29.

FIG. 42 A plan view for illustrating a process of fabricating the ultraviolet radiation sensor according to the fifth embodiment shown in FIG. 29.

FIG. 43 A sectional view for illustrating a process of fabricating the ultraviolet radiation sensor according to the fifth embodiment shown in FIG. 29.

FIG. 44 A plan view for illustrating a process of fabricating the ultraviolet radiation sensor according to the fifth embodiment shown in FIG. 29.

FIG. 45 A sectional view for illustrating a process of fabricating the ultraviolet radiation sensor according to the fifth embodiment shown in FIG. 29.

FIG. 46 A sectional view for illustrating a process of fabricating the ultraviolet radiation sensor according to the fifth embodiment shown in FIG. 29.

FIG. 47 A sectional view for illustrating a process of fabricating the ultraviolet radiation sensor according to the fifth embodiment shown in FIG. 29.

FIG. 48 A sectional view showing a field-effect transistor according to a sixth embodiment of the present invention.

FIG. 49 A sectional view for illustrating a process of fabricating the field-effect transistor according to the sixth embodiment shown in FIG. 48.

FIG. 50 A sectional view for illustrating a process of fabricating the field-effect transistor according to the sixth embodiment shown in FIG. 48.

FIG. 51 A sectional view for illustrating a process of fabricating the field-effect transistor according to the sixth embodiment shown in FIG. 48.

FIG. 52 A sectional view for illustrating a process of fabricating the field-effect transistor according to the sixth embodiment shown in FIG. 48.

FIG. 53 A sectional view for illustrating a process of fabricating the field-effect transistor according to the sixth embodiment shown in FIG. 48.

FIG. 54 A sectional view for illustrating a process of fabricating the field-effect transistor according to the sixth embodiment shown in FIG. 48.

FIG. 55 A graph showing an electric potential in a gate electrode in light-reception and in non-light reception.

FIG. 56 A plan view showing a modification of the ultraviolet radiation sensor according to the fifth embodiment shown in FIG. 29.

### BEST MODES FOR CARRYING OUT THE INVENTION

Embodiments of the present invention will be hereinafter described with reference to the drawings.

### First Embodiment

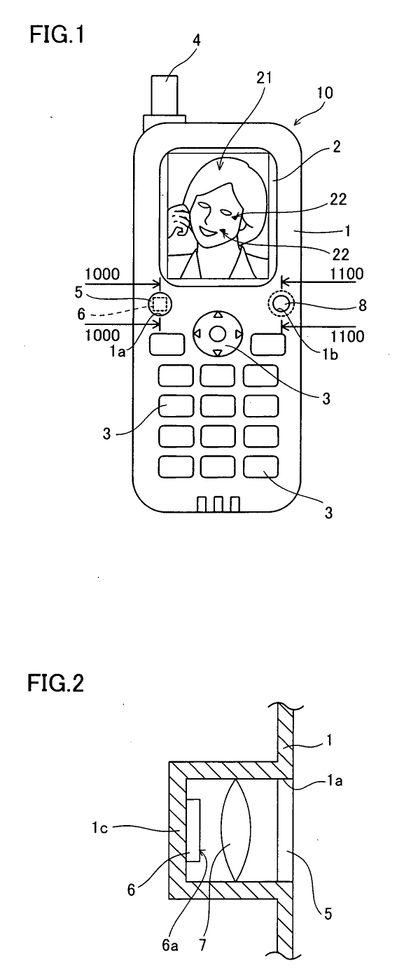

A structure of a cellular phone 10 employed as an information terminal (electric device) according to the first embodiment will be now described with reference to FIGS. 1 to 4.

This cellular phone 10 according to the first embodiment is so formed as to be capable of confirming sections where pigmented spots 22 on a skin of a human body 21 exist, as shown in FIG. 1. The human body 21 is an example of the “object” in the present invention, and the pigmented spot 22 is an example of the “black section” or the “section that is not black for a naked eye but absorbs ultraviolet radiation” in the present invention.

As a specific structure of the cellular phone 10 according to the first embodiment, a liquid crystal display 2 and a plurality of operation buttons 3 are provided in a housing 1. The liquid crystal display 2 is an example of the “display section” in the present invention. The liquid crystal display 2 is so arranged as to be exposed from the inside of the housing 1, and the operation buttons 3 are so arranged as to be exposed from the inside of the housing 1. The housing 1 is provided with an antenna 4 protruding from the inside to the outside of the housing 1. Additionally, the housing 1 is provided with two openings 1a and 1b and a mounting section 1c (see FIG. 2) for mounting a two-dimensional CCD (charge-coupled device) 6 described later is provided on a section corresponding to the opening 1a of the housing 1.

According to the first embodiment, an ultraviolet radiation filter 5, the two-dimensional CCD 6 and a lens 7 are arranged on a section corresponding to the opening 1a of the housing 1 as shown in FIGS. 1 and 2. The two-dimensional CCD 6 is an example of the “image detecting portion” in the present invention. More specifically, the ultraviolet radiation filter 5 is so mounted as to close the opening 1a of the housing 1. The two-dimensional CCD 6 includes a plurality of pixels (not shown) arranged two-dimensionally and is mounted on the mounting section 1c of the housing 1 such that light-receiving surfaces 6a of the respective pixels are opposed to the ultraviolet radiation filter 5. According to the first embodiment, an ultraviolet radiation sensor (not shown) is provided on at least one pixel among the plurality of pixels of the two-dimensional CCD 6. The lens 7 is mounted between the ultraviolet radiation filter 5 and the two-dimensional CCD 6.

According to the first embodiment, the ultraviolet radiation filter 5 is formed such that only ultraviolet radiation of at most about 400 nm is transmitted therethrough, and the lens 7 has a function of condensing ultraviolet radiation transmitted through the ultraviolet radiation filter 5 on the light-receiving surfaces 6a of the two-dimensional CCD 6. Thus, in this two-dimensional CCD 6 according to the first embodiment, only the ultraviolet radiation reflected on the skin of the human body 21 is incident upon the light-receiving surfaces 6a when imaging the human body 21, and hence an image of the human body 21 by ultraviolet radiation can be detected. This detected image of the human body 21 by the ultraviolet radiation is converted into electric signals to be outputted from the two-dimensional CCD 6.

The pigmented spots 22 on the skin of the human body 21 each have a property of absorbing the ultraviolet radiation and hence the reflectance of the ultraviolet radiation on sections where the pigmented spots 22 on the skin of the human body 21 exist is smaller than that of the ultraviolet radiation on a section where no pigmented spot 22 exists. Thus, the amount of the ultraviolet radiation incident upon the pixels corresponding to the sections where the pigmented spots 22 on the skin of the human body 21 exist is smaller than that of the ultraviolet radiation incident upon the pixels corresponding to the section where no pigmented spot 22 exists. Therefore, electric signals different from electric signals generated in the pixels corresponding to the section where no pigmented spot 22 exists are generated in the pixels corresponding to the sections where the pigmented spots 22 on the skin of the human body 21 exist.

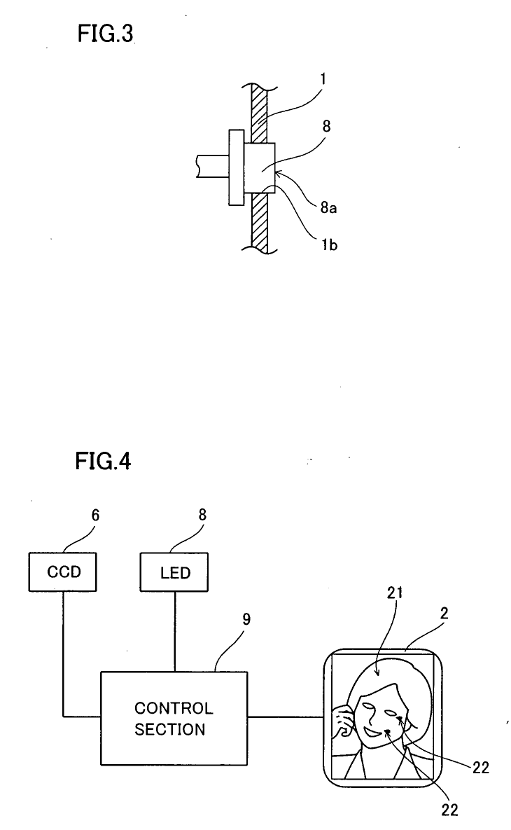

According to the first embodiment, an ultraviolet LED (light-emitting diode device) 8 emitting the ultraviolet radiation is mounted on the opening 1b of the housing 1 such that a light emission surface 8a protrudes to the outside of the housing 1, as shown in FIGS. 1 and 3. The ultraviolet LED 8 is an example of the “light-emitting portion” in the present invention. The light-emitting wavelength of the ultraviolet LED 8 is set to about 365 nm, and the intensity of the ultraviolet radiation emitted from the ultraviolet LED 8 is set to at most about 0.15 W/m². The image of the human body 21 by the ultraviolet radiation is detected with the two-dimensional CCD 6 by lighting the ultraviolet LED 8 also when imaging the human body 21 with the two-dimensional CCD 6 under an environment where the amount of the ultraviolet radiation is small (in a room or at night, for example).

According to the first embodiment, the liquid crystal display 2, the two-dimensional CCD 6 and the ultraviolet LED 8 are connected to a control section 9 constituted by a CPU, a ROM, a RAM and the like in the housing 1, as shown in FIG. 4. This control section 9 has a function of controlling an imaging operation of the two-dimensional CCD 6 and a light emitting operation of the ultraviolet LED 8. The control section 9 has a function of generating video signals corresponding to the image of the human body 21 by the ultraviolet radiation on the basis of the electric signals corresponding to the image of the human body 21 by the ultraviolet radiation generated with the two-dimensional CCD 6 and outputting the video signals to the liquid crystal display 2. Thus, the image of the human body 21 by the ultraviolet radiation is displayed on the liquid crystal display 2.

As hereinabove described, the electric signals generated in the pixels corresponding to the sections where the pigmented spots 22 on the skin of the human body 21 exist and the electric signals generated in the pixels corresponding to the section where no pigmented spot 22 exists are different from each other, and hence video signals corresponding to sections where the pigmented spots 22 on the skin of the human body 21 exist and video signals corresponding to a section where no pigmented spot 22 exists can be different from each other in the control section 9 according to the first embodiment. According to this first embodiment, the video signals are generated in the control section 9 such that the display color of the sections where the pigmented spots 22 on the skin of the human body 21 exist is black as compared with that of the section where no pigmented spot 22 exists.

An operation for displaying the image of the human body 21 by the ultraviolet radiation with the cellular phone 10 according to the first embodiment will be now described with reference to FIGS. 1 to 4.

A shooting mode is changed by operating the operation buttons 3 shown in FIG. 1, thereby bringing into a state capable of taking an image with the two-dimensional CCD 6. Then light emission mode (ON/OFF of an automatic light emission mode) of the ultraviolet LED 8 is set by operating the operation buttons 3. In a case where the automatic light emission mode is in an ON-state, the ultraviolet LED 8 is automatically lighted when an image is taken with the two-dimensional CCD 6 under an environment where the amount of the ultraviolet radiation is small. In a case where the automatic light emission mode is in an OFF-state, lighting the ultraviolet LED 8 can be manually controlled. Thereafter the image of the human body 21 is taken with the two-dimensional CCD 6 by operating the operation buttons 3.

At this time, according to the first embodiment, only ultraviolet radiation reflected on the skin of the human body 21 is transmitted through the ultraviolet radiation filter 5 and is incident upon the two-dimensional CCD 6. Thus, the image of the human body 21 by the ultraviolet radiation is detected in the two-dimensional CCD 6. The image of the human body 21 by the ultraviolet radiation is converted into the electric signals to be outputted from the two-dimensional CCD 6 to the control section 9 (see FIG. 4).

In the control section 9 shown in FIG. 4, the video signals are generated on the basis of the electric signals corresponding to the image of the human body 21 by the ultraviolet radiation and outputted to the liquid crystal display 2. Thus, the image of the human body 21 by the ultraviolet radiation is displayed on the liquid crystal display 2.

At this time, according to the first embodiment, when the pigmented spots 22 exist on the skin of the human body 21, the display color of the sections where the pigmented spots 22 on the skin of the human body 21 exist is black as compared with that of the section where no pigmented spot 22 exists. Thus, the sections where the pigmented spots 22 on the skin of the human body 21 exist can be confirmed when the pigmented spots 22 exist on the skin of the human body 21.

According to the first embodiment, as hereinabove described, the two-dimensional CCD 6 for detecting the image by the ultraviolet radiation reflecting the human body 21 by receiving the ultraviolet radiation reflected on the skin of the human body 21 and the liquid crystal display 2 for displaying the image by the ultraviolet radiation detected with the two-dimensional CCD 6 are provided, whereby when the image of the human body 21 by the ultraviolet radiation is detected with the two-dimensional CCD 6 and the image by the ultraviolet radiation is displayed on the liquid crystal display 2, the sections where the pigmented spots 22 on the skin of the human body 21 exist and the section where no pigmented spot 22 on the skin of the human body 21 exists are different from each other in the detectable amount of the ultraviolet radiation with the two-dimensional CCD 6, and hence the image of the human body 21 by the ultraviolet radiation can be displayed on the liquid crystal display 2 such that the display color of the sections where the pigmented spots 22 on the skin of the human body 21 exist and the display color of the section where no pigmented spot 22 exists are different from each other. Consequently, the sections where the pigmented spots 22 on the skin of the human body 21 exist can be confirmed with the cellular phone 10.

According to the first embodiment, as hereinabove described, the ultraviolet radiation filter 5 through which only the ultraviolet radiation is transmitted is arranged on the side closer to the light-receiving surfaces 6a of the two-dimensional CCD 6, whereby only the ultraviolet radiation transmitting through the ultraviolet radiation filter 5 is incident upon the light-receiving surface 6 of the two-dimensional CCD 6, and hence the two-dimensional CCD 6 can easily detect the image by the ultraviolet radiation.

According to the first embodiment, as hereinabove described, the ultraviolet LED 8 emitting the ultraviolet radiation is provided, whereby when the ultraviolet radiation is applied to the human body 21 by lighting the ultraviolet LED 8, the two-dimensional CCD 6 can detect the image of the human body 21 by the ultraviolet radiation also under the environment where the amount of the ultraviolet radiation is small (in a room or at night, for example).

Sapporo is a city where the amount of the ultraviolet radiation is the smallest in Japan, and winter is a season where the amount of the ultraviolet radiation is the smallest in a year. In winter in Sapporo, the amount of the ultraviolet radiation (wave UVB) having a wavelength of about 280 nm to about 320 nm irradiated from 10 o'clock to 14 o'clock (for about 14,400 seconds) is about 1500 Ws/m², and the average of the ultraviolet radiation intensity thereof during the period is about 0.10 Ws/m². The ultraviolet radiation (wave UVA) having a wavelength of about 320 nm to about 400 nm has an intensity (about 0.10 Ws/m²) of about five times that of the ultraviolet radiation (wave UVB) having the wavelength of about 280 nm to about 320 nm, and hence the average intensity of the ultraviolet radiation (wave UVA) having the wavelength of about 320 nm to about 400 nm during 10 o'clock to 14 o'clock in winter in Sapporo is about 0.5 Ws/m². In other words, the smallest intensity of the ultraviolet radiation (wave UVA) having the wavelength of about 320 nm to about 400 nm is about 0.5 Ws/m² in nature.

According to the first embodiment where the intensity of the ultraviolet radiation (wavelength: about 365 nm) emitted from the ultraviolet LED 8 is set to at most about 0.15 Ws/m², the intensity (about 0.15 Ws/m²) of the ultraviolet radiation emitted from the ultraviolet LED 8 is smaller than the intensity (about 0.5 Ws/m²) of the ultraviolet radiation having the wavelength of about 320 nm to about 400 nm in nature, and hence immunity of the human body 21 can be inhibited from being disadvantageously reduced due to application of the ultraviolet radiation to the human body 21 by lighting the ultraviolet LED 8.

Referring to FIG. 5, according to a first modification of this first embodiment, the ultraviolet radiation filter 5, the two-dimensional CCD 6 and the lens 7 shown in FIG. 2 are arranged on a section corresponding to an opening 31a of a housing 31 of a personal digital assistant (information terminal) 30 dissimilarly to the aforementioned first embodiment. An ultraviolet LED 8 shown in FIG. 3 is arranged on a section corresponding to an opening 31b of the housing 31 of the personal digital assistant 30.

A liquid crystal display 32 displaying an image by ultraviolet radiation is so provided in the housing 31 as to be exposed from the inside of the housing 31. The liquid crystal display 32 is an example of the “display section” in the present invention. Operation buttons 33 are so provided in the housing 31 as to be exposed from the inside of the housing 31. A shooting mode or a light emission mode is changed by operating the operation buttons 33 and an image is taken with the two-dimensional CCD 6.

An inner structure of the personal digital assistant 30 is similar to that of the cellular phone 10 according to the first embodiment shown in FIG. 4.

According to the aforementioned structure, in the personal digital assistant 30 according to the first modification of the first embodiment, the image of the human body 21 by the ultraviolet radiation can be displayed on the liquid crystal display 32 such that the display color of sections where pigmented spots 22 on a skin of a human body 21 exist and the display color of a section where no pigmented spot 22 exists are different from each other, similarly to the aforementioned first embodiment. Thus, the sections where the pigmented spots 22 on the skin of the human body 21 exist can be confirmed with the personal digital assistant 30.

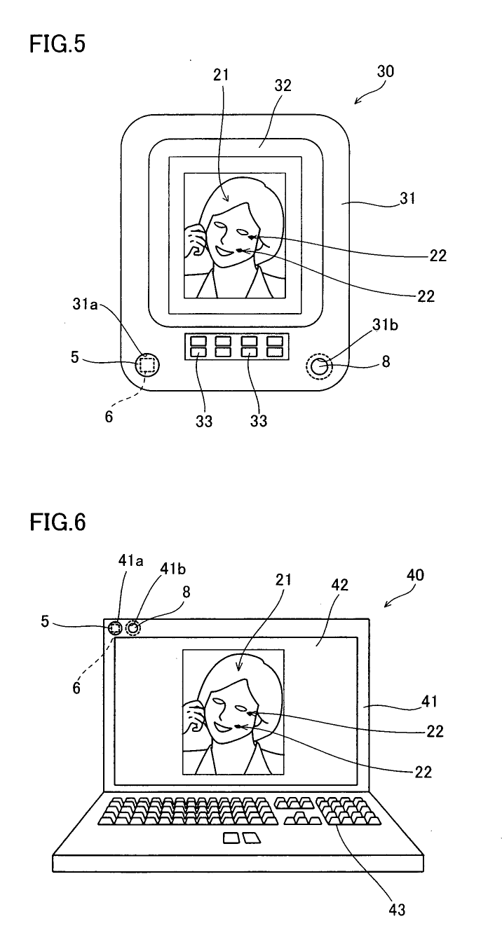

Referring to FIG. 6, according to a second modification of the first embodiment, the ultraviolet radiation filter 5, the two-dimensional CCD 6 and the lens 7 shown in FIG. 2 are arranged on a section corresponding to an opening 41a of a housing 41 of a laptop personal computer (information terminal) 40, dissimilarly to the aforementioned first embodiment. The ultraviolet LED 8 shown in FIG. 3 is arranged on a section corresponding to an opening 41b of the housing 41 of the laptop personal computer 40.

A liquid crystal display 42 displaying an image by ultraviolet radiation is so provided in the housing 41 as to be exposed from the inside of the housing 41. The liquid crystal display 42 is an example of the “display section” in the present invention. A keyboard 43 is so provided in the housing 41 as to be exposed from the inside of the housing 41. A shooting mode or a light emission mode is changed by operating the keyboard 43 and an image is taken with the two-dimensional CCD 6.

An inner structure of the laptop personal computer 40 is similar to that of the cellular phone 10 according to the first embodiment shown in FIG. 4.

According to the aforementioned structure, in the laptop personal computer 40 according to the second modification of the first embodiment, the image of the human body 21 by the ultraviolet radiation can be displayed on the liquid crystal display 42 such that the display color of sections where pigmented spots 22 on a skin of a human body 21 exist and the display color of a section where no pigmented spot 22 exists are different from each other, similarly to the aforementioned first embodiment. Thus, the sections where the pigmented spots 22 on the skin of the human body 21 exist can be confirmed with the laptop personal computer 40.

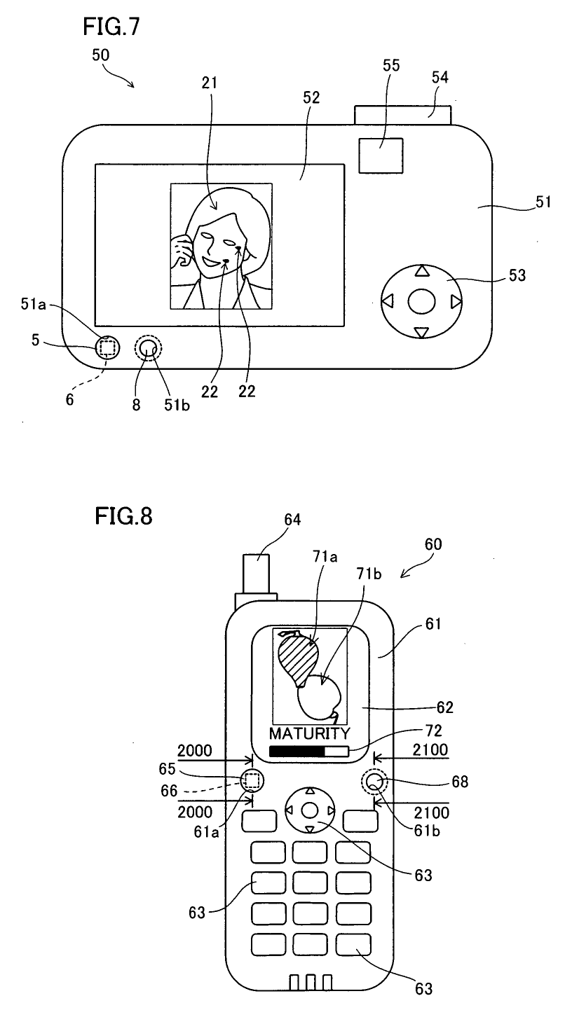

Referring to FIG. 7, according to a third modification of the first embodiment, the ultraviolet radiation filter 5, the two-dimensional CCD 6 and the lens 7 shown in FIG. 2 are arranged on a section corresponding to an opening 51a of a housing 51 of a digital camera (electronic still camera) (information terminal) 50, dissimilarly to the aforementioned first embodiment. The ultraviolet LED 8 shown in FIG. 3 is arranged on a section corresponding to an opening 51b of the housing 51 of the digital camera 50.

A liquid crystal display 52 displaying an image by ultraviolet radiation is so provided in the housing 51 as to be exposed from the inside of the housing 51. The liquid crystal display 52 is an example of the “display section” in the present invention. Operation buttons 53 are so provided in the housing 51 as to be exposed from the inside of the housing 51. A shooting mode or a light emission mode is changed by operating the operation buttons 53. A shutter 54 is provided in the housing 51 such that one end thereof protrudes upwardly. An image is taken with the two-dimensional CCD 6 by operating this shutter 54. A viewfinder 55 usually employed in the shooting mode is provided on the housing 51.

An inner structure of the digital camera 50 is similar to that of the cellular phone 10 according to the first embodiment shown in FIG. 4.

According to the aforementioned structure, in the digital camera 50 according to the third modification of the first embodiment, the image of the human body 21 by the ultraviolet radiation can be displayed on the liquid crystal display 52 such that the display color of sections where pigmented spots 22 on a skin of a human body 21 exist and the display color of a section where no pigmented spot 22 exists are different from each other, similarly to the aforementioned first embodiment. Thus, the sections where the pigmented spots 22 on the skin of the human body 21 exist can be confirmed with the digital camera 50.

### Second Embodiment

In this second embodiment, a case of distinguishing between a vegetable 71a containing a large quantity of antioxidant substances (maturity is high) and a vegetable 71b containing a small quantity of antioxidant substances (maturity is low) will be described with reference to FIGS. 8 to 11, dissimilarly to the aforementioned first embodiment.

A cellular phone (information terminal) 60 according to this second embodiment is so formed as to be capable of distinguishing between the vegetable 71a containing the large quantity of antioxidant substances and the vegetable 71b containing the small quantity of antioxidant substances, as shown in FIG. 8. The vegetables 71a and 71b are each an example of the “object” in the present invention.

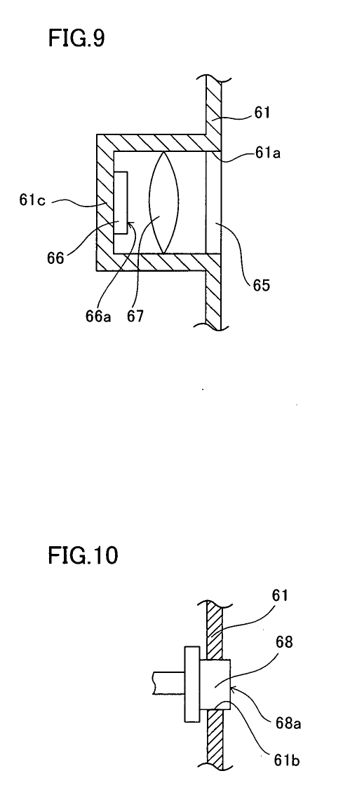

As a specific structure of the cellular phone 60 according to the second embodiment, a liquid crystal display 62 and a plurality of operation buttons 3 are provided in a housing 61. The liquid crystal display 62 is an example of the “display section” in the present invention. The liquid crystal display 62 is so arranged as to be exposed from the inside of the housing 61, and the operation buttons 63 are so arranged as to be exposed from the inside of the housing 61. The housing 61 is provided with an antenna 64 protruding from the inside to the outside of the housing 61. Additionally, the housing 61 is provided with two openings 61a and 61b and a mounting section 61c (see FIG. 9) for mounting a two-dimensional CCD (charge-coupled device) 66 described later is provided on a section corresponding to the opening 61a of the housing 61.

According to the second embodiment, an ultraviolet radiation filter 5, the two-dimensional CCD 6 and a lens 7 are arranged on a section corresponding to the opening 61a of the housing 61 as shown in FIGS. 8 and 9. The two-dimensional CCD 66 is an example of the “image detecting portion” in the present invention. More specifically, the ultraviolet radiation filter 65 is so mounted as to close the opening 61a of the housing 61. The two-dimensional CCD 66 includes a plurality of pixels (not shown) arranged two-dimensionally and is mounted on the mounting section 61c of the housing 61 such that light-receiving surfaces 66a of the respective pixels are opposed to the ultraviolet radiation filter 65. According to the second embodiment, an ultraviolet radiation sensor (not shown) is provided on at least one pixel among the plurality of pixels of the two-dimensional CCD 66. The lens 67 is mounted between the ultraviolet radiation filter 65 and the two-dimensional CCD 66.

According to the second embodiment, the ultraviolet radiation filter 65 is formed such that only ultraviolet radiation of at most about 400 nm is transmitted therethrough, and the lens 67 has a function of condensing ultraviolet radiation transmitted through the ultraviolet radiation filter 65 on the light-receiving surfaces 66a of the two-dimensional CCD 66. Thus, in this two-dimensional CCD 66 according to the second embodiment, only the ultraviolet radiation reflected on surfaces of the vegetables 71a and 71b is incident upon the light-receiving surfaces 66a when imaging the vegetables 71a and 71b, and hence images of the vegetables 71a and 71b by ultraviolet radiation can be detected. These detected images of the vegetables 71a and 71b by the ultraviolet radiation are converted into electric signals to be outputted from the two-dimensional CCD 66.

The antioxidant substances (polyphenol, flavone, flavonol, anthocyanin, lutein, chlorophyll and the like) contained in the vegetables 71a and 71b each have a property of absorbing the ultraviolet radiation, and hence the reflectance of the ultraviolet radiation on the surface of the vegetable 71a containing the large quantity of antioxidant substances is smaller than that of the ultraviolet radiation on the surface of the vegetable 71b containing the small quantity of antioxidant substances. Thus, the amount of the ultraviolet radiation incident upon the pixels corresponding to the vegetable 71a containing the large quantity of antioxidant substances is smaller than that of the ultraviolet radiation incident upon the pixels corresponding to the vegetable 71b containing the small quantity of antioxidant substances. Therefore, electric signals different from electric signals generated in the pixels corresponding to the vegetable 71a containing the large quantity of antioxidant substances are generated in the pixels corresponding to the vegetable 71b containing the small quantity of antioxidant substances.

According to the second embodiment, an ultraviolet LED (light-emitting diode device) 68 emitting the ultraviolet radiation is mounted on an opening 61b of the housing 61 such that a light emission surface 68a protrudes to the outside of the housing 61, as shown in FIGS. 8 and 10. The ultraviolet LED 68 is an example of the “light-emitting portion” in the present invention. The light-emitting wavelength of the ultraviolet LED 68 is set to about 365 nm, and the intensity of the ultraviolet radiation emitted from the ultraviolet LED 68 is set to at most about 0.15 W/m². The images of the vegetables 71a and 71b by the ultraviolet radiation are detected with the two-dimensional CCD 66 by lighting the ultraviolet LED 68 also when imaging the vegetables 71a and 71b with the two-dimensional CCD 66 under an environment where the amount of the ultraviolet radiation is small (in a room or at night, for example).

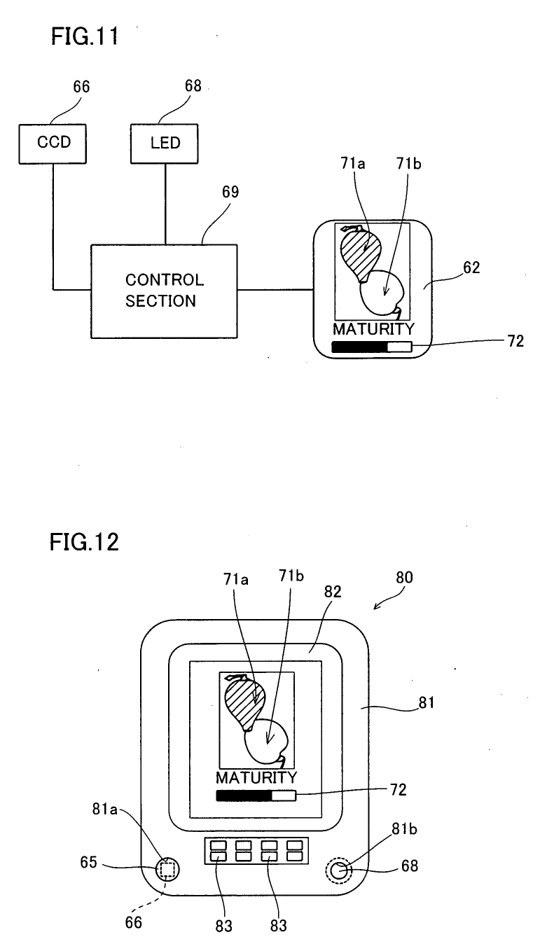

According to the second embodiment, the liquid crystal display 62, the two-dimensional CCD 66 and the ultraviolet LED 68 are connected to a control section 69 constituted by a CPU, a ROM, a RAM and the like in the housing 61, as shown in FIG. 11. This control section 69 has a function of controlling an imaging operation of the two-dimensional CCD 66 and a light emitting operation of the ultraviolet LED 68. The control section 69 has a function of generating video signals corresponding to the images of the vegetables 71a and 71b by the ultraviolet radiation on the basis of the electric signals corresponding to the images of the vegetables 71a and 71b by the ultraviolet radiation generated with the two-dimensional CCD 66 and outputting the video signals to the liquid crystal display 62. Thus, the images of the vegetables 71a and 71b by the ultraviolet radiation are displayed on the liquid crystal display 62.

As hereinabove described, the electric signals generated in the pixels corresponding to the vegetable 71a containing the large quantity of antioxidant substances and the electric signals generated in the pixels corresponding to the vegetable 71b containing the small quantity of antioxidant substances are different from each other, and hence the video signals corresponding to the vegetable 71a containing the large quantity of antioxidant substances and the video signals corresponding to the vegetable 71b containing the small quantity of antioxidant substances can be different from each other in the control section 69 according to the second embodiment. According to this second embodiment, the video signals are generated in the control section 69 such that the display color of the vegetable 71a containing the large quantity of antioxidant substances is black as compared with that of the vegetable 71b containing the small quantity of antioxidant substances.

According to the second embodiment, the control section 69 is so formed as to be capable of calculating the maturity of either the vegetable 71a or 71b. The maturity of the vegetable 71a or 71b is displayed on the liquid crystal display 62 with a bar graph 72.

According to the second embodiment, maturity M (%) of the vegetable 71a or 71b is calculated with the control section 69 according to the following expression (1):

M=((S_{AR}-S_{R})/S_{AR})×100 (1)

S_{AR} in the aforementioned expression (1) represents the intensity of electric signals obtained by converting ultraviolet radiation by the ultraviolet radiation sensor of the two-dimensional CCD 66 in a case where it has been assumed that all the ultraviolet radiation is not absorbed but reflected on the surface of the vegetable 71a (71b). S_{R} in the aforementioned expression (1) represents the intensity of electric signals obtained by converting the ultraviolet radiation actually reflected on the surface of the vegetable 71a (71b) by the ultraviolet radiation sensor of the two-dimensional CCD 66.

It has been known that increase in the quantity of antioxidant substances heightens the maturity in vegetable 71a (71b) containing the antioxidant substances (polyphenol, flavone, flavonol, anthocyanin, lutein, chlorophyll and the like) absorbing the ultraviolet radiation. In other words, the reflectance of the ultraviolet radiation on the surface of the vegetable 71a having high maturity is smaller than that of the ultraviolet radiation on the surface of the vegetable 71b having low maturity due to the contained large quantity of antioxidant substances. Therefore, the intensity of the electric signals obtained by converting the ultraviolet radiation reflected on the surface of the vegetable 71a having the high maturity is smaller than that of the electric signal obtained by converting the ultraviolet radiation reflected on the surface of the vegetable 71b having the low maturity. For example, the maturity M of the vegetable 71a having the high maturity is 70%, and the maturity M of the vegetable 71b having the low maturity is 30%, assuming that the S_{AR} in the case where the ultraviolet radiation is not absorbed but reflected on the surface of the vegetable 71a (71b) is 1, the S_{R} in the vegetable 71a having the high maturity (containing the large quantity of antioxidant substances) is 0.3, and the S_{R} in the vegetable 71b having the low maturity (containing the small quantity of antioxidant substances) is 0.7 in the aforementioned expression (1).

An operation for displaying the images of the vegetables 71a and 71b by the ultraviolet radiation with the cellular phone 60 according to the second embodiment will be now described with reference to FIGS. 8 to 11.

A shooting mode is changed by operating the operation buttons 63 shown in FIG. 8, thereby bringing into a state capable of taking an image with the two-dimensional CCD 66. Then light emission mode (ON/OFF of an automatic light emission mode) of the ultraviolet LED 68 is set by operating the operation buttons 63. In a case where the automatic light emission mode is in an ON-state, the ultraviolet LED 68 is automatically lighted when an image is taken with the two-dimensional CCD 66 under an environment where the amount of the ultraviolet radiation is small. In a case where the automatic light emission mode is in an OFF-state, lighting the ultraviolet LED 68 can be manually controlled. Thereafter the images of the vegetables 71a and 71b are taken with the two-dimensional CCD 66 by operating the operation buttons 63.

At this time, according to the second embodiment, only ultraviolet radiation reflected on the surfaces of the vegetables 71a and 71b is transmitted through the ultraviolet radiation filter 65 and is incident upon the two-dimensional CCD 66. Thus, the images of the vegetables 71a and 71b by the ultraviolet radiation are detected in the two-dimensional CCD 66. The images of the vegetables 71a and 71b by the ultraviolet radiation are converted into the electric signals to be outputted from the two-dimensional CCD 66 to the control section 69 (see FIG. 11).

In the control section 69 shown in FIG. 11, the video signals are generated on the basis of the electric signals corresponding to the images of the vegetables 71a and 71b by the ultraviolet radiation and outputted to the liquid crystal display 62. Thus, the images of the vegetables 71a and 71b by the ultraviolet radiation are displayed on the liquid crystal display 62.

At this time, according to the second embodiment, the display color of the vegetable 71a containing the large quantity of antioxidant substances is black as compared with that of the vegetable 71b containing the small quantity of antioxidant substances. Thus, the vegetable 71a containing the large quantity of antioxidant substances and the vegetable 71b containing the small quantity of antioxidant substances can be distinguished from each other. According to the second embodiment, the maturity of either the vegetable 71a containing the large quantity of antioxidant substances or the vegetable 71b containing the small quantity of antioxidant substances is displayed on the liquid crystal display 62 with the bar graph 72.

According to the second embodiment, as hereinabove described, the two-dimensional CCD 66 for detecting the images by the ultraviolet radiation reflecting the vegetables 71a and 71b by receiving the ultraviolet radiation reflected on the surfaces of the vegetables 71a and 71b and the liquid crystal display 62 for displaying the images by the ultraviolet radiation detected with the two-dimensional CCD 66 are provided, whereby when the images of the vegetables 71a and 71b by the ultraviolet radiation is detected with the two-dimensional CCD 66 and the images by the ultraviolet radiation are displayed on the liquid crystal display 62, the vegetable 71a containing the large quantity of antioxidant substances and the vegetable 71b containing the small quantity of antioxidant substances are different from each other in the detectable amount of the ultraviolet radiation with the two-dimensional CCD 66, and hence the images of the vegetables 71a and 71b by the ultraviolet radiation can be displayed on the liquid crystal display 62 such that the display color of the vegetable 71a containing the large quantity of antioxidant substances and the display color of the vegetable 71b containing the small quantity of antioxidant substances are different from each other. Consequently, the vegetable 71a containing the large quantity of antioxidant substances (maturity is high) and the vegetable 71b containing the small quantity of antioxidant substances (maturity is low) can be distinguished from each other with the cellular phone 60.

According to the second embodiment, as hereinabove described, the maturity of the vegetable 71a or 71b is displayed on the liquid crystal display 62 with the bar graph 72, whereby the maturity of the vegetable 71a having the high maturity or the vegetable 71b having the low maturity can be easily confirmed.

According to the second embodiment, as hereinabove described, the ultraviolet radiation filter 65 through which only the ultraviolet radiation is transmitted is arranged on the side closer to the light-receiving surfaces 66a of the two-dimensional CCD 66, whereby only the ultraviolet radiation transmitting through the ultraviolet radiation filter 65 is incident upon the light-receiving surface 66 of the two-dimensional CCD 66, and hence the two-dimensional CCD 66 can easily detect the images by the ultraviolet radiation.

According to the second embodiment, as hereinabove described, the ultraviolet LED 68 emitting the ultraviolet radiation is provided, whereby when the ultraviolet radiation is applied to the vegetables 71a and 71b by lighting the ultraviolet LED 68, the two-dimensional CCD 66 can detect the images of the vegetables 71a and 71b by the ultraviolet radiation also under the environment where the amount of the ultraviolet radiation is small (in a room or at night, for example).

Referring to FIG. 12, according to a first modification of this second embodiment, the ultraviolet radiation filter 65, the two-dimensional CCD 66 and the lens 67 shown in FIG. 9 are arranged on a section corresponding to an opening 81a of a housing 81 of a personal digital assistant (information terminal) 80 dissimilarly to the aforementioned second embodiment. An ultraviolet LED 68 shown in FIG. 10 is arranged on a section corresponding to an opening 81b of the housing 81 of the personal digital assistant 80.

A liquid crystal display 82 displaying images by ultraviolet radiation is so provided in the housing 81 as to be exposed from the inside of the housing 81. The liquid crystal display 82 is an example of the “display section” in the present invention. Operation buttons 83 are so provided in the housing 81 as to be exposed from the inside of the housing 81. A shooting mode or a light emission mode is changed by operating the operation buttons 83 and an image is taken with the two-dimensional CCD 66.

An inner structure of the personal digital assistant 80 is similar to that of the cellular phone 60 according to the second embodiment shown in FIG. 11.

According to the aforementioned structure, in the personal digital assistant 80 according to the first modification of the second embodiment, the images of the vegetables 71a and 71b by the ultraviolet radiation can be displayed on the liquid crystal display 82 such that the display color of the vegetable 71a containing the large quantity of antioxidant substances and the display color of the vegetable 71b containing the small quantity of antioxidant substances are different from each other, similarly to the aforementioned second embodiment. Thus, the vegetable 71a containing the large quantity of antioxidant substances (maturity is high) and the vegetable 71b containing the small quantity of antioxidant substances (maturity is low) can be distinguished from each other with the personal digital assistant 80.

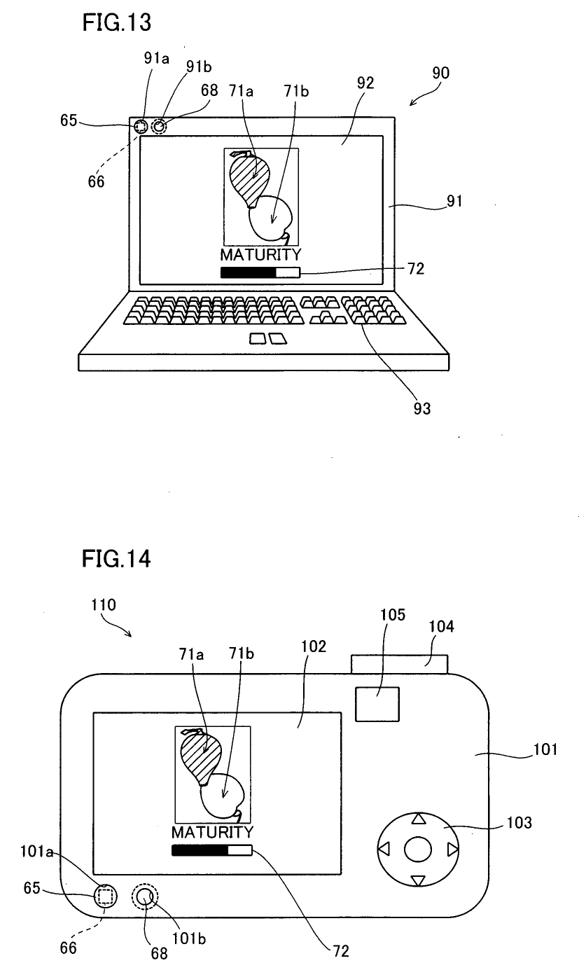

Referring to FIG. 13, according to a second modification of the second embodiment, the ultraviolet radiation filter 65, the two-dimensional CCD 66 and the lens 67 shown in FIG. 9 are arranged on a section corresponding to an opening 91a of a housing 91 of a laptop personal computer (information terminal) 90, dissimilarly to the aforementioned second embodiment. The ultraviolet LED 68 shown in FIG. 10 is arranged on a section corresponding to an opening 91b of the housing 91 of the laptop personal computer 90.

A liquid crystal display 92 displaying an image by ultraviolet radiation is so provided in the housing 91 as to be exposed from the inside of the housing 91. The liquid crystal display 92 is an example of the “display section” in the present invention. A keyboard 93 is so provided in the housing 91 as to be exposed from the inside of the housing 91. A shooting mode or a light emission mode is changed by operating the keyboard 93 and an image is taken with the two-dimensional CCD 66.

An inner structure of the laptop personal computer 90 is similar to that of the cellular phone 10 according to the second embodiment shown in FIG. 11.

According to the aforementioned structure, in the laptop personal computer 90 according to the second modification of the second embodiment, the images of the vegetables 71a and 71b by the ultraviolet radiation can be displayed on the liquid crystal display 92 such that the display color of the vegetable 71a containing the large quantity of antioxidant substances and the display color of the vegetable 71b containing the small quantity of antioxidant substances are different from each other, similarly to the aforementioned second embodiment. Thus, the vegetable 71a containing the large quantity of antioxidant substances (maturity is high) and the vegetable 71b containing a small quantity of antioxidant substances (maturity is low) can be distinguished from each other with the laptop personal computer 90.

Referring to FIG. 14, according to a third modification of the second embodiment, the ultraviolet radiation filter 65, the two-dimensional CCD 66 and the lens 67 shown in FIG. 9 are arranged on a section corresponding to an opening 101a of a housing 101 of a digital camera (electronic still camera) (information terminal) 110, dissimilarly to the aforementioned second embodiment. The ultraviolet LED 68 shown in FIG. 10 is arranged on a section corresponding to an opening 101b of the housing 101 of the digital camera 110.

A liquid crystal display 102 displaying an image by ultraviolet radiation is so provided in the housing 101 as to be exposed from the inside of the housing 111. The liquid crystal display 102 is an example of the “display section” in the present invention. Operation buttons 103 are so provided in the housing 101 as to be exposed from the inside of the housing 101. A shooting mode or a light emission mode is changed by operating the operation buttons 103. A shutter 104 is provided in the housing 101 such that one end thereof protrudes upwardly. An image is taken with the two-dimensional CCD 66 by operating this shutter 104. A viewfinder 105 usually employed in the shooting mode is provided on the housing 101.

An inner structure of the digital camera 110 is similar to that of the cellular phone 60 according to the second embodiment shown in FIG. 11.

According to the aforementioned structure, in the digital camera 110 according to the third modification of the second embodiment, the images of the vegetables 71a and 71b by the ultraviolet radiation can be displayed on the liquid crystal display 102 such that the display color of the vegetable 71a containing the large quantity of antioxidant substances and the display color of the vegetable 71b containing the small quantity of antioxidant substances are different from each other, similarly to the aforementioned second embodiment. Thus, the vegetable 71a containing the large quantity of antioxidant substances (maturity is high) and the vegetable 71b containing the small quantity of antioxidant substances (maturity is low) can be distinguished from each other with the digital camera 110.

### Third Embodiment

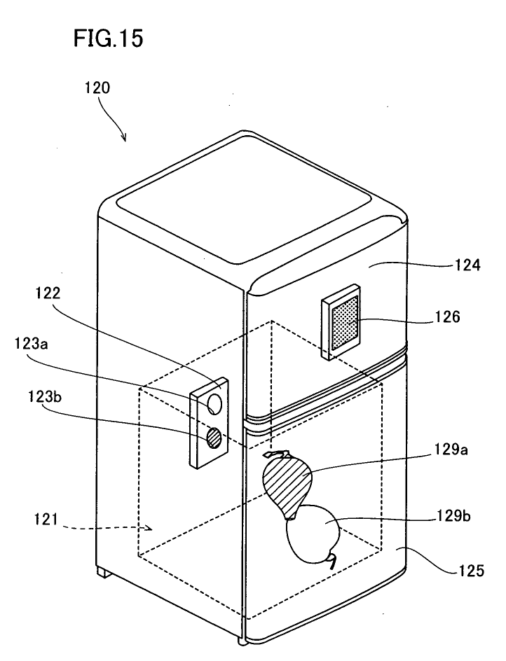

A structure of an electric refrigerator (electric device) 120 according to a third embodiment will be now described with reference to FIGS. 15 to 19.

The electric refrigerator 120 according to this third embodiment comprises a vegetable compartment 121 controlling inside thereof at about 5° C., as shown in FIG. 15. The vegetable compartment 121 is an example of the “storage section” in the present invention. The vegetable compartment 121 has a protruding section 122 on a side surface thereof, and openings 123a and 123b are provided on a surface of the protruding section 122. The openings 123a and 123b are provided with a two-dimensional CCD (charge-coupled device) 127 and an ultraviolet LED 128 described later, respectively. The electric refrigerator 120 has a refrigeration compartment door 124 and a vegetable compartment door 125, a liquid crystal display 126 is provided on a surface of the refrigeration compartment door 124. Vegetables 129a and 129b are stored in the vegetable compartment 121. The liquid crystal display 126 is an example of the “display section” in the present invention, and the vegetables 129a and 129b are each an example of the “object” in the present invention.

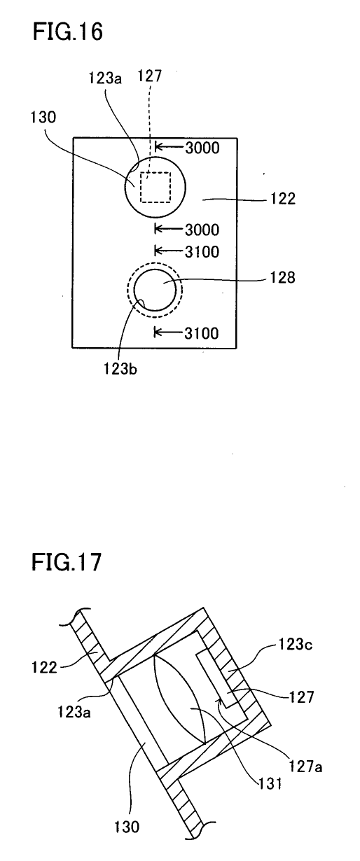

According to the third embodiment, a protective filter 130, the two-dimensional CCD 127 and a lens 131 are arranged on a section corresponding to the opening 123a as shown in FIGS. 16 and 17. The two-dimensional CCD 127 is an example of the “image detecting portion” in the present invention. More specifically, the protective filter 130 is so mounted as to close the opening 123a of the protruding section 122. The two-dimensional CCD 127 includes a plurality of pixels (not shown) arranged two-dimensionally and is mounted on a mounting section 123c integral with the protruding section 122 such that light-receiving surfaces 127a of the respective pixels are opposed to the protective filter 130. According to the third embodiment, an ultraviolet radiation sensor (not shown) is provided on at least one pixel among the plurality of pixels of the two-dimensional CCD 127. The lens 131 is mounted between the protective filter 130 and the two-dimensional CCD 127. An ultraviolet radiation filter may be employed in place of the protective filter 130. When this ultraviolet radiation filter is formed such that only ultraviolet radiation of at most about 400 nm is transmitted therethrough, only the ultraviolet radiation can be incident upon the two-dimensional CCD 127 also in a case where visible light enters inside the vegetable compartment 121.

According to the third embodiment, the lens 131 has a function of condensing ultraviolet radiation transmitted through the protective filter 130 on the light-receiving surfaces 127a of the two-dimensional CCD 127. Thus, in this two-dimensional CCD 127 according to the third embodiment, only the ultraviolet radiation reflected on the vegetables 129a and 129b is incident upon the light-receiving surfaces 127a when imaging the vegetables 129a and 129b in the vegetable compartment 121, and hence images of the vegetables 129a and 129b by ultraviolet radiation can be detected. The detected images of the vegetables 129a and 129b by the ultraviolet radiation are converted into electric signals and to be outputted from the two-dimensional CCD 127. In the two-dimensional CCD 127, a dark current is suppressed under a low-temperature environment such as in the vegetable compartment 121, whereby detection accuracy of the ultraviolet radiation can be increased.

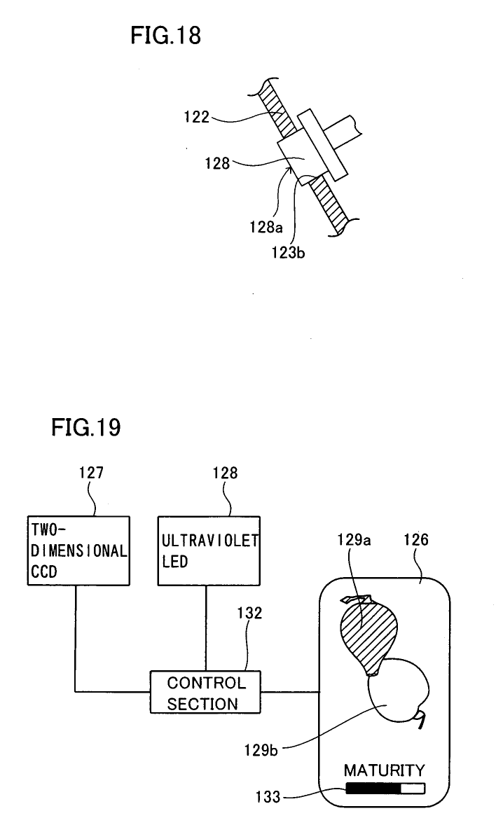

According to the third embodiment, an ultraviolet LED (light-emitting diode device) 128 emitting the ultraviolet radiation is mounted on the opening 123b of the protruding section 122 such that a light emission surface 128a protrudes to the outside of the protruding section 122, as shown in FIGS. 16 and 18. The ultraviolet LED 128 is an example of the “light-emitting portion” in the present invention. The light-emitting wavelength of the ultraviolet LED 128 is set to about 365 nm, and the intensity of the ultraviolet radiation emitted from the ultraviolet LED 128 is set to at most about 0.15 W/m². The images of the vegetables 129a and 129b by the ultraviolet radiation are detected in the closed vegetable compartment 121 where no visible light exists with the two-dimensional CCD 127 by lighting the ultraviolet LED 128. The ultraviolet LED 128 is capable of stabilizing a light output under the low-temperature environment such as in the vegetable compartment 121.

According to the third embodiment where the intensity of the ultraviolet radiation (wavelength: about 365 nm) emitted from the ultraviolet LED 128 is set to at most about 0.15 Ws/m², the intensity (about 0.15 Ws/m²) of the ultraviolet radiation emitted from the ultraviolet LED 128 is smaller than the intensity (about 0.5 Ws/m²) of the ultraviolet radiation having the wavelength of about 320 nm to about 400 nm in nature described in the aforementioned first embodiment, and hence immunity of the human body can be inhibited from being disadvantageously reduced due to possible application of the ultraviolet radiation to the human body by lighting the ultraviolet LED 128.

The antioxidant substances (polyphenol, flavone, flavonol, anthocyanin, lutein, chlorophyll and the like) contained in vegetables and fruits each have a property of absorbing the ultraviolet radiation, and hence the reflectance of the ultraviolet radiation on the surface of the vegetable 129a (see FIG. 15) containing the large quantity of antioxidant substances is smaller than that of the ultraviolet radiation on the vegetable 129b containing the small quantity of antioxidant substances. Thus, the amount of the ultraviolet radiation incident upon the pixels corresponding to the vegetable 129a containing the large quantity of antioxidant substances is smaller than that of the ultraviolet radiation incident upon the pixels corresponding to the vegetable 129b containing the small quantity of antioxidant substances. Therefore, electric signals different from electric signals generated in the pixels corresponding to the vegetable 129a containing the large quantity of antioxidant substances are generated in the pixels corresponding to the vegetable 129b containing the small quantity of antioxidant substances.

As shown in FIG. 19, the liquid crystal display 126, the two-dimensional CCD 127 and the ultraviolet LED 128 are connected to a control section 132 constituted by a CPU, a ROM, a RAM and the like in protruding section 122 (see FIG. 15). This control section 132 has a function of controlling an imaging operation of the two-dimensional CCD 127 and a light emitting operation of the ultraviolet LED 128. The control section 132 has a function of generating video signals corresponding to the images of the vegetables 129a and 129b by the ultraviolet radiation on the basis of the electric signals corresponding to the images of the vegetables 129a and 129b by the ultraviolet radiation generated with the two-dimensional CCD 127 and outputting the video signals to the liquid crystal display 126. Thus, the images of the vegetables 129a and 129b by the ultraviolet radiation are displayed on the liquid crystal display 126.

As hereinabove described, the electric signals generated in the pixels corresponding to the vegetable 129a containing the large quantity of antioxidant substances and the electric signals generated in the pixels corresponding to the vegetable 129b containing the small quantity of antioxidant substances are different from each other, and hence the video signals corresponding to the vegetable 129a containing the large quantity of antioxidant substances and the video signals corresponding to the vegetable 129b containing the small quantity of antioxidant substances can be different from each other in the control section 132 according to the second embodiment. According to this third embodiment, the video signals are generated in the control section 132 such that the display color of the vegetable 129a containing the large quantity of antioxidant substances is black as compared with that of the vegetable 129b containing the small quantity of antioxidant substances.

According to the third embodiment, the control section 132 is so formed as to be capable of calculating the maturity of either the vegetable 129a or 129b. The maturity of the vegetable 129a or 129b is displayed on the liquid crystal display 126 with an indicator 133.

According to the third embodiment, maturity M (%) of the vegetable 129a or 129b is calculated with the control section 132 according to the expression (1) described in the aforementioned first embodiment:

According to the third embodiment, as hereinabove described, the two-dimensional CCD 127 for detecting the images by the ultraviolet radiation reflecting the vegetables 129a and 129b stored in the vegetable compartment 121 by receiving the ultraviolet radiation reflected on the surfaces of the vegetables 129a and 129b stored in the vegetable compartment 121 and the liquid crystal display 126 for displaying the images by the ultraviolet radiation detected with the two-dimensional CCD 127 are provided, whereby when the images of the vegetables 129a and 129b stored in the vegetable compartment 121 by the ultraviolet radiation are detected with the two-dimensional CCD 127 and the images by the ultraviolet radiation are displayed on the liquid crystal display 126, the vegetable 129a containing the large quantity of antioxidant substances and the vegetable 129b containing the small quantity of antioxidant substances are different from each other in the detectable amount of the ultraviolet radiation with the two-dimensional CCD 127, and hence the images of the vegetables 129a and 129b by the ultraviolet radiation can be displayed on the liquid crystal display 126 such that the display color of the vegetable 129a containing the large quantity of antioxidant substances is deeper than the display color of the vegetable 129b containing the small quantity of antioxidant substances. Consequently, the vegetable 129a containing the large quantity of antioxidant substances (maturity is high) stored in the vegetable compartment 121 and the vegetable 129b containing the small quantity of antioxidant substances (maturity is low) stored in the vegetable compartment 121 can be distinguished from each other without opening the vegetable compartment door 125 of the electric refrigerator 120.

According to the third embodiment, as hereinabove described, the maturity of the vegetable 129a or 129b is displayed on the liquid crystal display 126 with the indicator 133, whereby the maturity of the vegetable 129a having the high maturity or the vegetable 129b having the low maturity can be easily confirmed.

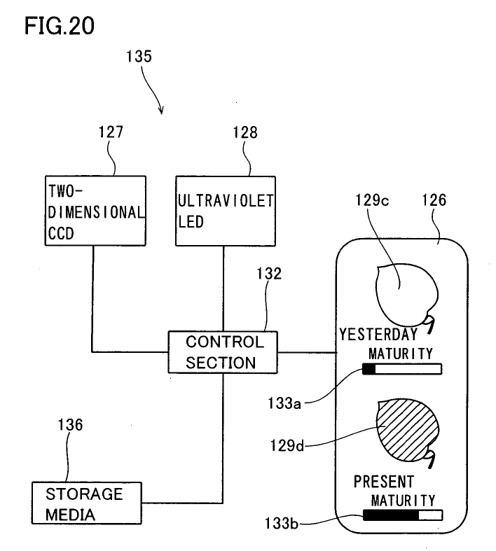

Referring to FIG. 20, according to a modification of this third embodiment, a storage media 136 such as a hard disk for storing an image by ultraviolet radiation is connected to a control section 132, dissimilarly to the aforementioned third embodiment. The structure of an electric refrigerator 135 and the remaining inner structure thereof are similar to those of the electric refrigerator 120 according to the third embodiment. The storage media 136 is an example of the “storage portion” in the present invention.

In the electric refrigerator 135 according to the modification of the third embodiment, images by ultraviolet radiation are stored in the storage media 136, whereby not only a vegetable 129d as an present image by the ultraviolet radiation but also a vegetable 129c as a past image by the ultraviolet radiation can be displayed on a liquid crystal display 126. Therefore, temporal change (temporal change of maturity) of the quantity of antioxidant substances of the same food can be confirmed, and hence arbitrary peak ripeness of the food can be easily estimated. The maturity of the past vegetable 129c is displayed on the liquid crystal display 126 with an indicator 133a and the maturity of the present vegetable 129d is displayed on the liquid crystal display 126 with an indicator 133b, whereby the temporal change (temporal change of maturity) of the maturity of the same food can be easily confirmed.

### Fourth Embodiment

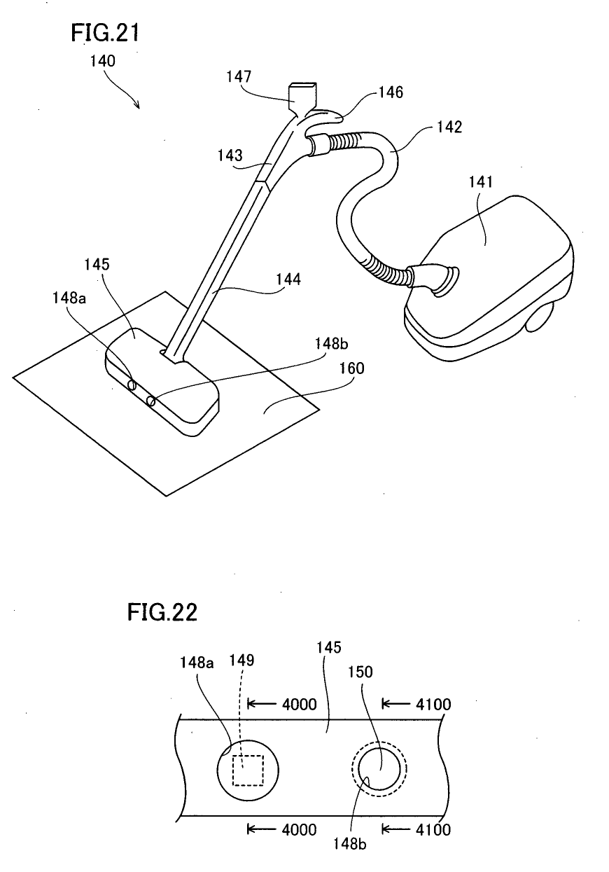

A structure of an electric vacuum cleaner (electric device) 140 according to a fourth embodiment will be described with reference to FIGS. 21 to 26.

The electric vacuum cleaner 140 according to this fourth embodiment comprises a cleaner object 141. The cleaner object 141 has a dust chamber (not shown) inside thereof and a first end of a hose 142 having flexibility is connected to a hose inlet leading to the dust chamber. A second end of the hose 142 is connected to a suction head 145 through a hard connecting pipe 143 and an extension pipe 144 of the connecting pipe 143 continuously. A grip section 146 gripped with a hand of an operator when cleaning is formed integrally on an upper surface of the connecting pipe 143. A liquid crystal display 147 displaying ultraviolet radiation information is provided on an upper surface of the grip section 146 such that a surface displaying the information is directed toward the operator. Two openings 148a and 148b are provided on a side surface section of the suction head 145 and a two-dimensional CCD (charge-coupled device) 149 and an ultraviolet LED 150 described later are mounted on the openings 148a and 148b respectively. The side surface section of the suction head 145 having the openings 148a and 148b is tapered such that the two-dimensional CCD 149 can receive reflection of ultraviolet radiation from the floor surface 160. This floor surface 160 is made of a flooring material, a carpet or the like. The liquid crystal display 147 is an example of the “display section” in the present invention. The floor surface 160 is an example of the “prescribed region” and the “cleaned region” in the present invention.

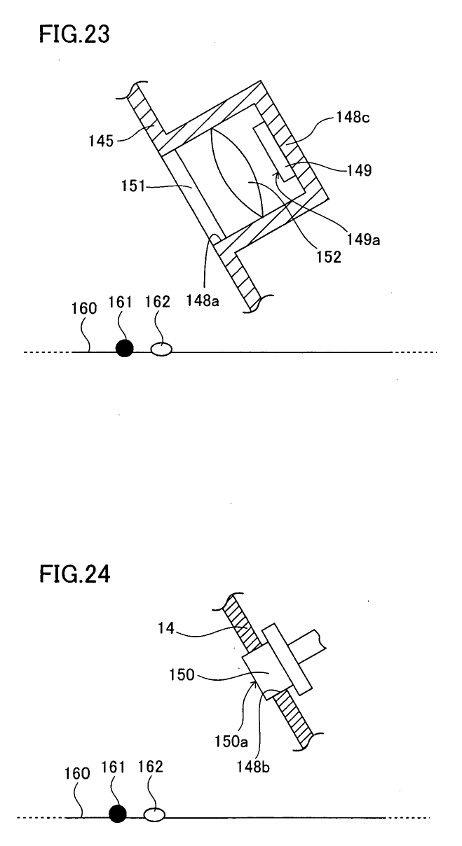

According to the fourth embodiment, an ultraviolet radiation filter 151, the two-dimensional CCD 149 and a lens 152 are arranged on a section corresponding to the opening 148a as shown in FIGS. 22 and 23. The two-dimensional CCD 149 is an example of the “image detecting portion” in the present invention. More specifically, the ultraviolet radiation filter 151 is so mounted as to close the opening 148a of the suction head 145. The two-dimensional CCD 149 includes a plurality of pixels (not shown) arranged two-dimensionally and is mounted on a mounting section 148c integral with the suction head 145 such that light-receiving surfaces 149a of the respective pixels are opposed to the ultraviolet radiation filter 151. According to the fourth embodiment, an ultraviolet radiation sensor (not shown) is provided on at least one pixel among the plurality of pixels of the two-dimensional CCD 149. The lens 152 is mounted between the ultraviolet radiation filter 151 and the two-dimensional CCD 149.

According to the fourth embodiment, the ultraviolet radiation filter 151 is formed such that only ultraviolet radiation of at most about 400 nm is transmitted therethrough, and the lens 152 has a function of condensing ultraviolet radiation transmitted through the ultraviolet radiation filter 151 on the light-receiving surfaces 149a of the two-dimensional CCD 149. Thus, in this two-dimensional CCD 149 according to the fourth embodiment, only the ultraviolet radiation reflected on the floor surface 160 is incident upon the light-receiving surfaces 149a when imaging the floor surface 160, and hence an image of the floor surface 160 by ultraviolet radiation can be detected. This detected image of the floor surface 160 by the ultraviolet radiation is converted into electric signals to be outputted from the two-dimensional CCD 149.

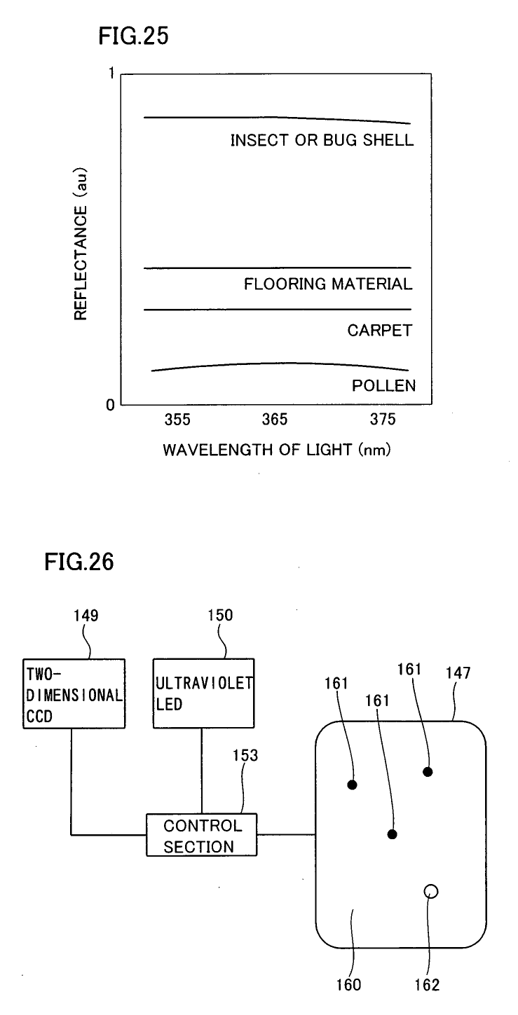

As shown in FIG. 25, a pollen 161 on the floor surface 160 has a property of absorbing the ultraviolet radiation and hence the reflectance of the ultraviolet radiation on a region where the pollen 161 on the floor surface 160 exists is smaller than that of the ultraviolet radiation on a region where no pollen 161 exists. Thus, the amount of the ultraviolet radiation incident upon the pixels corresponding to the region where the pollen 161 on the floor surface 160 exists is smaller than that of the ultraviolet radiation incident upon the pixels corresponding to the region where no pollen 161 exists. Therefore, electric signals different from electric signals generated in the pixels corresponding to the region where no pollen 161 exists are generated in the pixels corresponding to the region where the pollen 161 on the floor surface 160 exists.

As shown in FIG. 25, an insect 162 or a bug shell thereof on the floor surface 160 has a property of reflecting the ultraviolet radiation and hence the reflectance of the ultraviolet radiation on a region where the insect 162 or the bug shell thereof on the floor surface 160 exists is larger than that of the ultraviolet radiation on a region where no insect 162 or no bug shell thereof exists. Thus, the amount of the ultraviolet radiation incident upon the pixels corresponding to the region where the insect 162 or the bug shell thereof on the floor surface 160 exists is larger than that of the ultraviolet radiation incident upon the pixels corresponding to the region where no insect 162 or no bug shell thereof exists. Therefore, electric signals different from electric signals generated in the pixels corresponding to the region where no insect 162 or no bug shell thereof exists are generated in the pixels corresponding to the region where the insect 162 or the bug shell thereof on the floor surface 160 exists. The insect 162 is a microorganism existing on a flooring material and a carpet such as a spider or a tick, for example.

According to the fourth embodiment, the ultraviolet LED (light-emitting diode device) 150 emitting the ultraviolet radiation is mounted on an opening 148b of the suction head 145 such that a light emission surface 150a protrudes to the outside of the suction head 145, as shown in FIGS. 21 and 24. The ultraviolet LED 150 is an example of the “light-emitting portion” in the present invention. The light-emitting wavelength of the ultraviolet LED 150 is set to about 365 nm, and the intensity of the ultraviolet radiation emitted from the ultraviolet LED 150 is set to at most about 0.15 W/m². The image of the floor surface 160 by the ultraviolet radiation is detected with the two-dimensional CCD 149 by lighting the ultraviolet LED 150 also when imaging the floor surface 160 with the two-dimensional CCD 149 under an environment where the amount of the ultraviolet radiation is small (in a room or at night, for example).

The liquid crystal display 147, the two-dimensional CCD 149 and the ultraviolet LED 150 are connected to a control section 153 constituted by a CPU, a ROM, a RAM and the like inside the grip section 146 (see FIG. 21), as shown in FIG. 26. This control section 153 has a function of controlling an imaging operation of the two-dimensional CCD 149 and a light emitting operation of the ultraviolet LED 150. The control section 153 has a function of generating video signals corresponding to the image of the floor surface 160 by the ultraviolet radiation on the basis of the electric signals corresponding to the image of the floor surface 160 by the ultraviolet radiation generated with the two-dimensional CCD 149 and outputting the video signals to the liquid crystal display 147. Thus, the image of the floor surface 160 by the ultraviolet radiation is displayed on the liquid crystal display 147.

As hereinabove described, the electric signals generated in the pixels corresponding to the region where the pollen 161, the insect 162 or the bug shell thereof on the floor surface 160 exists and the electric signals generated in the pixels corresponding to the region where none of the pollen 161 and the insect 162 or the bug shell thereof exist are different from each other, and hence video signals corresponding to the region where the pollen 161, the insect 162 or the bug shell thereof on the floor surface 160 exists and video signals corresponding to the region where none of the pollen 161 and the insect 162 or the bug shell thereof exist can be different from each other in the control section 153 according to the fourth embodiment. According to this fourth embodiment, the video signals are generated in the control section 153 such that the display color of the region where the pollen 161 on the floor surface 160 exists is black as compared with that of the region where no pollen 161 exists and the region where the insect 162 or the bug shell thereof on the floor surface 160 exists is white as compared with the display color of the region where the no insect 162 or no bug shell pollen 161 exists, respectively.

According to the fourth embodiment, as hereinabove described, the two-dimensional CCD 149 for detecting the image by the ultraviolet radiation reflecting the floor surface 160 by receiving the ultraviolet radiation reflected on the floor surface 160 and the liquid crystal display 147 for displaying the image by the ultraviolet radiation detected with the two-dimensional CCD 149 are provided, whereby when the image of the floor surface 160 by the ultraviolet radiation is detected with the two-dimensional CCD 149 and the image by the ultraviolet radiation is displayed on the liquid crystal display 147, the region where the pollen 161, the insect 162 or the bug shell thereof on the floor surface 160 exists and the region where none of the pollen 161 and the insect 162 or the bug shell thereof exist are different from each other in the detectable amount of the ultraviolet radiation with the two-dimensional CCD 149, and hence the image of the floor surface 160 by the ultraviolet radiation can be displayed on the liquid crystal display 147 such that the display color of the region where the pollen 161, the insect 162 or the bug shell thereof on the floor surface 160 exists and the display color of the region where none of the pollen 161 and the insect 162 or the bug shell thereof exist are different from each other. Consequently, the region where the pollen 161, the insect 162 or the bug shell thereof on the floor surface 160 exists can be confirmed and hence the pollen 161, the insect 162 or the bug shell thereof can be reliably cleaned.

The remaining effects of the fourth embodiment are similar to those of the aforementioned first embodiment.

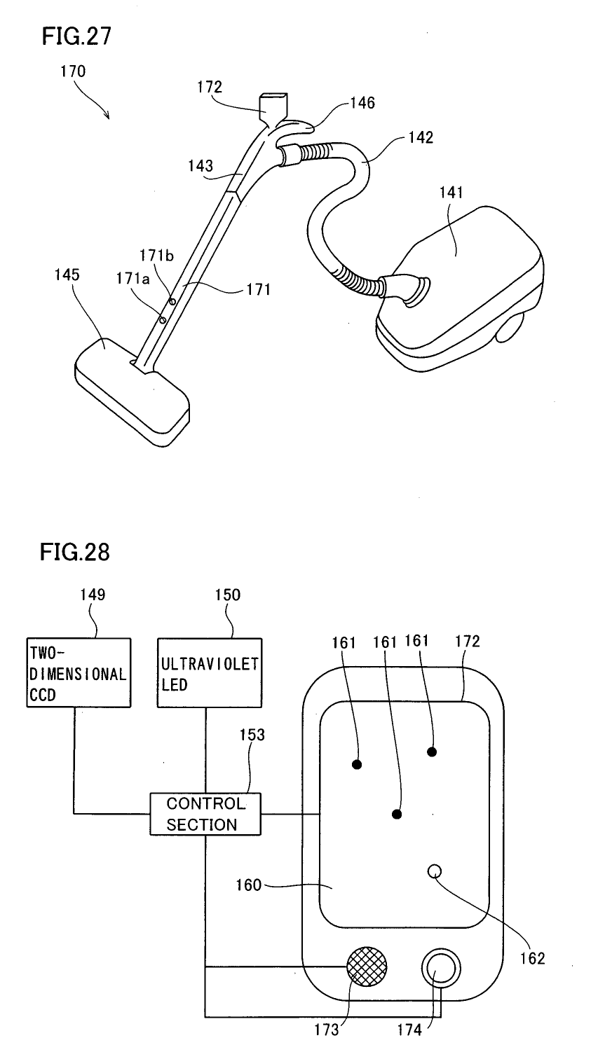

Referring to FIGS. 27 and 28, according to a modification of this fourth embodiment, the ultraviolet radiation filter 151, the two-dimensional CCD 149 and the lens 152 shown in FIG. 23 are arranged on a portion corresponding to an opening 171a of an extension pipe 171 of an electric vacuum cleaner 170 dissimilarly to the aforementioned fourth embodiment. An ultraviolet LED 150 shown in FIG. 24 is arranged on a portion corresponding to an opening 171b of the extension pipe 171 of the electric vacuum cleaner 170. As shown in FIG. 28, a buzzer 173 and a visible light LED 174 are provided on a liquid crystal display 172. The buzzer 173 and the visible light LED 174 are examples of the “first annunciation portion” and the “second annunciation portion” in the present invention respectively, and the liquid crystal display 172 is an example of the “display section” in the present invention. The buzzer 173 and the visible light LED 174 are connected to the control section 153. The control section 153 has a function of operating the buzzer 173 and the visible light LED 174 when detecting a different electric signal on the basis of the electric signal of each pixel of the image of the floor surface 160 by the ultraviolet radiation generated with the two-dimensional CCD 149.

In the electric vacuum cleaner 170 according to the modification of the fourth embodiment, the two-dimensional CCD 149 is provided on the portion corresponding to the opening 171a of the extension pipe 171, whereby the distance from the floor surface 160 to the two-dimensional CCD 149 can be increased and hence a wider range of the image of the floor surface 160 can be imaged. Consequently, the wider range of the image of the floor surface 160 can be displayed on the liquid crystal display 172. The buzzer 173 and the visible light LED 174 are provided, whereby the electric signal different from the electric signal of each pixel generated in the two-dimensional CCD 149 is generated due to variation in the ultraviolet radiation reflectance when the pollen 161, the insect 162 or the bug shell hereof exists on the floor surface 160, and hence the control section 153 detecting it plays the sounds of the buzzer 173 and emits the light of the visible light LED 174. Thus, it is possible to announce the existence of the pollen 161, the insect 162 or the bug shell thereof to the operator, and hence the operator does not need to always monitor the liquid crystal display 172.

### Fifth Embodiment

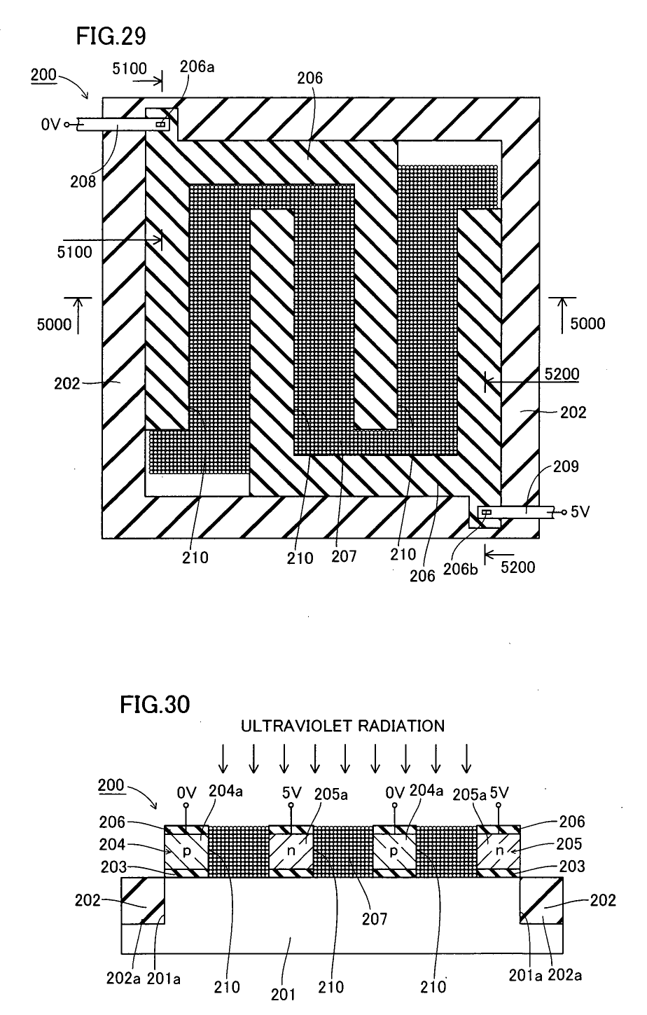

A structure of an ultraviolet radiation sensor 200 according to the fifth embodiment of the present invention will be now described with reference to FIGS. 29 to 36.

The ultraviolet radiation sensor 200 according to the fifth embodiment comprises an n-type of p-type silicon substrate 201 as shown in FIG. 30. The silicon substrate 201 is an example of the “substrate” or the “conductive substrate” in the present invention. As shown in FIGS. 29 and 30, an element isolation region 202 formed by STI (shallow trench isolation) having a structure in which an insulating film 202a is embedded in an element isolation groove 201a formed on the silicon substrate 201 is so formed on a prescribed region of a surface of the silicon substrate 201 as to surround an element forming region. Insulating layers 203 made of SiO₂ each having a thickness of about 2 nm to about 10 nm is formed on prescribed regions of the surface of the silicon substrate 201 in the element forming region surrounded by the element isolation region 202. These insulating layers 203 for insulating a p-type and n-type polysilicon layers 204 and 205 and the silicon substrate 201 are provided on regions corresponding to forming regions of the p-type and n-type polysilicon layer 204 and 205 described later.

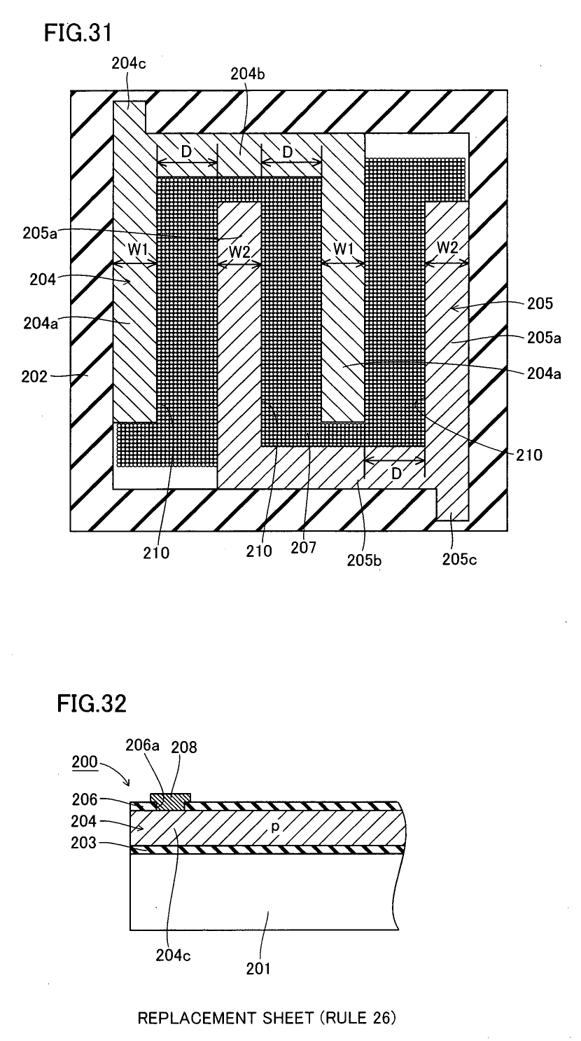

According to the fifth embodiment, the p-type and n-type polysilicon layers 204 and 205 each having a thickness of about 50 nm to about 200 nm are formed on upper surfaces of the insulating layers 203 at prescribed intervals in a horizontal direction. These p-type and n-type polysilicon layers 204 and 205 each have a function as an electrode. The p-type polysilicon layer 204 is an example of the “first electrode” or the “p-type semiconductor layer” in the present invention, and the n-type polysilicon layer 205 is an example of the “second electrode” and the “n-type semiconductor layer” in the present invention. As shown in FIG. 31, the p-type polysilicon layer 204 includes two electrode sections 204a and one coupling section 204b coupling the two electrode sections 204a. Thus the p-type polysilicon layer 204 is formed in a U-shape (comb-shape) in plan view by the electrode sections 204a and the coupling section 204b. Similarly, the n-type polysilicon layer 205 includes two electrode sections 205a and one coupling section 205b coupling the two electrode sections 205a, and is formed in the U-shape (comb-shape) in plan view. Each of the electrode sections 204a of the p-type polysilicon layer 204 and each of the electrode sections 205a of the n-type polysilicon layer 205 have widths W1 and W2 each of about 0.1 μm to about 0.5 μm, respectively. Each electrode section 204a of the p-type polysilicon layer 204 and each electrode section 205a of the n-type polysilicon layer 205 are so arranged as to be opposed at an interval D of about 0.1 μm to about 1.0 μm. In other words, three grooves 210 each having a width (interval D) of about 0.1 μm to about 1.0 μm are provided between the electrode sections 204a of the p-type polysilicon layer 204 and the electrode sections 205a of the n-type polysilicon layer 205. The p-type and n-type polysilicon layers 204 and 205 are provided with contact sections 204c and 205c for electrically connecting voltage supply electrodes 208 and 209 (see FIG. 29) made of aluminum respectively.

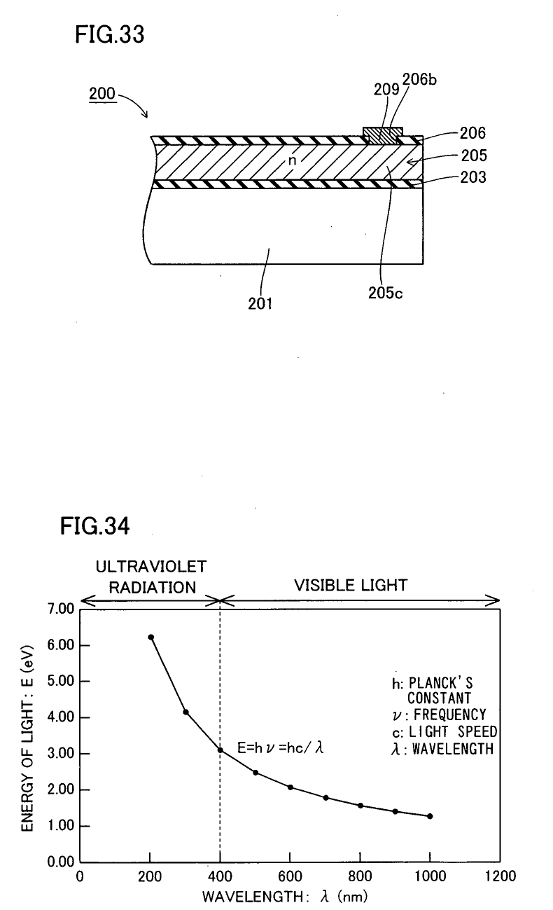

As shown in FIGS. 29 and 30, insulating layers 206 made of SiO₂ having a thickness of about 5 nm to about 50 nm are provided on upper surfaces of the p-type and n-type polysilicon layers 204 and 205. The insulating layers 206 are provided for insulating the surfaces of the p-type and n-type polysilicon layers 204 and 205. As shown in FIG. 32, a contact hole 206a for electrically connecting the voltage supply electrode 208 to the p-type polysilicon layer 204 is provided on a region of the insulating layer 206 corresponding to the contact section 204c of the p-type polysilicon layer 204. As shown in FIG. 33, a contact hole 206b for electrically connecting the voltage supply electrode 209 to the n-type polysilicon layer 205 is provided on a region of the insulating layer 206 corresponding to the contact section 205c of the n-type polysilicon layer 205. Voltages of about 0 V and about 5 V are applied to the p-type and n-type pblysilicon layers 204 and 205 respectively.

According to the fifth embodiment, silicon nanoparticle layers 207 made of silicon nanoparticles are embedded in grooves 210 between the electrode sections 204a of the p-type polysilicon layer 204 and the electrode sections 205a of the n-type polysilicon layer 205 arranged at the prescribed horizontal intervals, as shown in FIGS. 29 and 30. The silicon nanoparticle layer 207 is an example of the “semiconductor layer” in the present invention. The silicon nanoparticles of the silicon nanoparticle layers 207 each have a particle side (about 1 nm) capable of having a band gap of about at least 3.1 eV. “Thin Film Silicon Nanoparticle UV Photodetector”, O. M. Nayfeh, et al., PHOTONICS TECHNOLOGY LETTER, VOL. 16, NO. 8, August 2004, PP 1927-1929, for example, discloses that particles each having a particle size of about 1 nm have a band gap of about 3 eV. Therefore, electrons are excited from the silicon nanoparticles when light having energy of at least about 3.1 eV is applied to the silicon nanoparticles having a band gap of about 3.1 eV. More specifically, light energy E is defined by a Planck's constant h, light speed c and wavelength λ and ultraviolet radiation of at most about 400 nm has energy of at least about 3.1 eV as shown in FIG. 34, and hence electrons are excited from the silicon nanoparticles when the silicon nanoparticle layers 207 receive the ultraviolet radiation. On the other hand, visible light having a wavelength longer than about 400 nm has energy smaller than that of about 3.1 eV and hence electrons are not excited from the silicon nanoparticles when the silicon nanoparticle layers 207 receive the visible light.

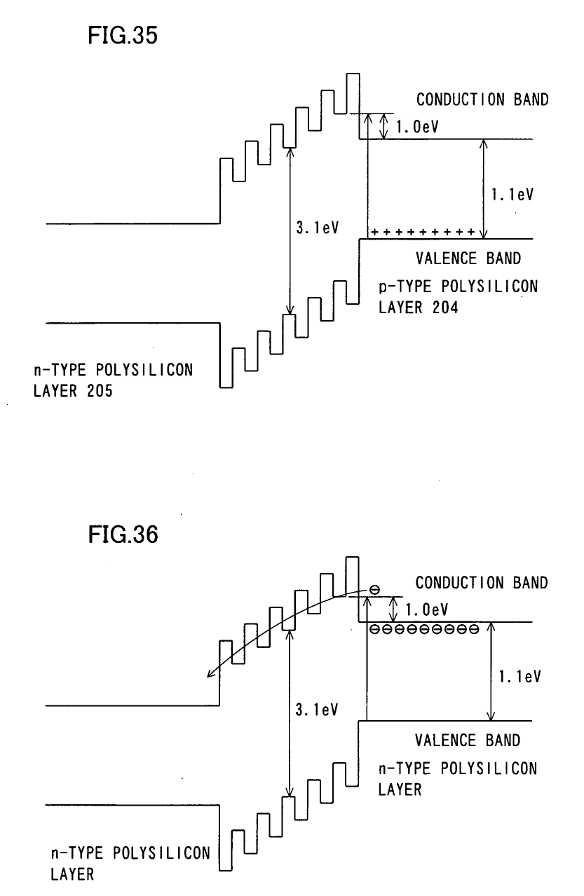

According to the fifth embodiment, in the structure where about 0 V is applied to the p-type polysilicon layer 204 and about 5 V is applied to the n-type polysilicon layer 205, electrons are required to be excited to the energy level from the valence band of the p-type polysilicon layer 204 to the conduction band of the silicon nanoparticles of the silicon nanoparticle layers 207 in order to excite electrons taking a role as a current from the p-type polysilicon layer 204 where the quantity of electrons are small on a conduction band, as shown in FIG. 35. Therefore, the energy (about 1.1 eV) on the band gap of the p-type polysilicon layer 204 and the energy (about 1.0 eV) up to the energy level on the conduction band of the silicon nanoparticles are required to be provided to the electron on the valence band of the p-type polysilicon layer 204 in order to excite the electrons on the valence band of the p-type polysilicon layer 204 to the energy level of the conduction band of the silicon nanoparticles of the silicon nanoparticle layers 207. When the visible light having a wavelength longer (energy smaller) than that of the ultraviolet radiation, electrons can be inhibited from being excited from the p-type polysilicon layer 204. Thus, the electrons excited by the visible light are gravitated to the n-type polysilicon layer 205 having a high potential (about 5 V) and therefore can be inhibited from being detected as a current. Consequently, only electrons excited by the ultraviolet radiation can be detected as a current, and hence detection accuracy of the ultraviolet radiation can be improved. In a structure where two n-type polysilicon layers are employed as electrodes as a comparative example, on the other hand, electrons are simply excited to the energy level up to the conduction band of the silicon nanoparticles of the silicon nanoparticle layers 207 from the conduction band of the n-type polysilicon layer in order to excite electrons taking a role as a current from the n-type polysilicon layer where the quantity of electrons are large on the conductive band, as shown in FIG. 36. In this case, only the energy (about 1.0 eV) up to the energy level of the conduction band of the silicon nanoparticles is simply provided to the electrons on the conduction band of the n-type polysilicon layer in order to excite the electrons on the conduction band of the n-type polysilicon layer up to the energy level of the conduction band of the silicon nanoparticles of the silicon nanoparticle layers 207. Thus, electrons are disadvantageously easily excited by small energy provided by the visible light when the visible light having a wavelength longer (energy smaller) than the ultraviolet radiation is incident upon the n-type polysilicon layer where the quantity of electrons are large on the conduction band. Therefore, the electrode are preferably formed by the p-type polysilicon layer 204 and the n-type polysilicon layer 205 as in the fifth embodiment as compared with the electrode formed by the two n-type polysilicon layers as in the comparative example.

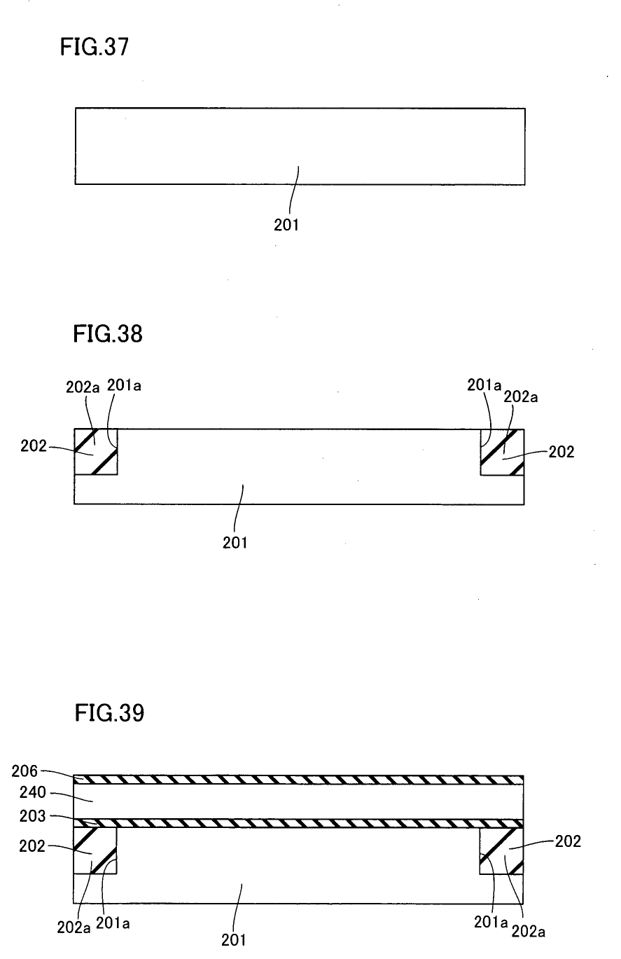

A process of fabricating the ultraviolet radiation sensor 200 according to the fifth embodiment will be now described with reference to FIGS. 37 to 47.

As shown in FIG. 37, the n-type or p-type silicon substrate 201 is prepared. As shown in FIG. 38, the element isolation groove 201a is so formed as to surround the element forming region on the prescribed region of the surface of the silicon substrate 201 by photolithography and etching. Then the element isolation insulating film 202a is so formed as to be embedded in the element isolation groove 201a of the silicon substrate 201 by thermal oxidation or CVD (chemical vapor deposition) and CMP (chemical mechanical polishing), thereby forming the element isolation region 202 formed by STI.

As shown in FIG. 39, the insulating layers 203 of SiO₂ each having a thickness of about 2 nm to 10 nm is formed on the upper surface of the silicon substrate 201 by thermal oxidation or CVD. A non-doped polysilicon layer 240 having a thickness of about 50 nm to about 200 nm is formed on the insulating layers 203 by CVD. Thereafter an insulating layer 206 made of SiO₂ having a thickness of about 50 nm to 200 nm is formed on the upper surface of the non-doped polysilicon layer 240 by CVD.

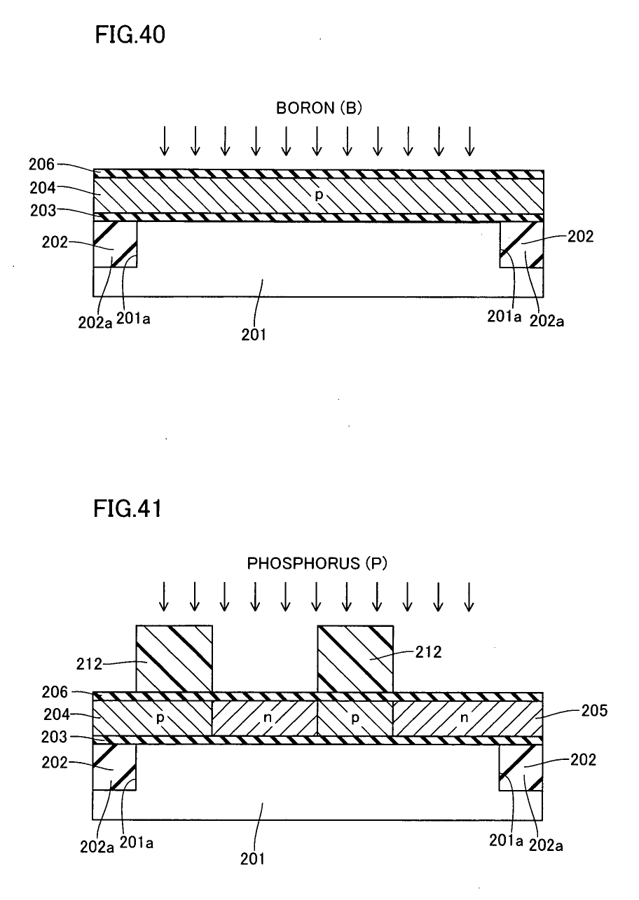

As shown in FIG. 40, boron (B) is ion-implanted into the non-doped polysilicon layer 240 (see FIG. 39) through the insulating film 206 under a condition of implantation energy of about 50 keV and a dose (implantation dosage) of about 1×10⁻¹⁵ cm⁻² to about 1×10⁻¹⁵ cm⁻². Thus, the non-doped polysilicon layer 240 is converted to the p-type, thereby forming the p-type polysilicon layer 204.

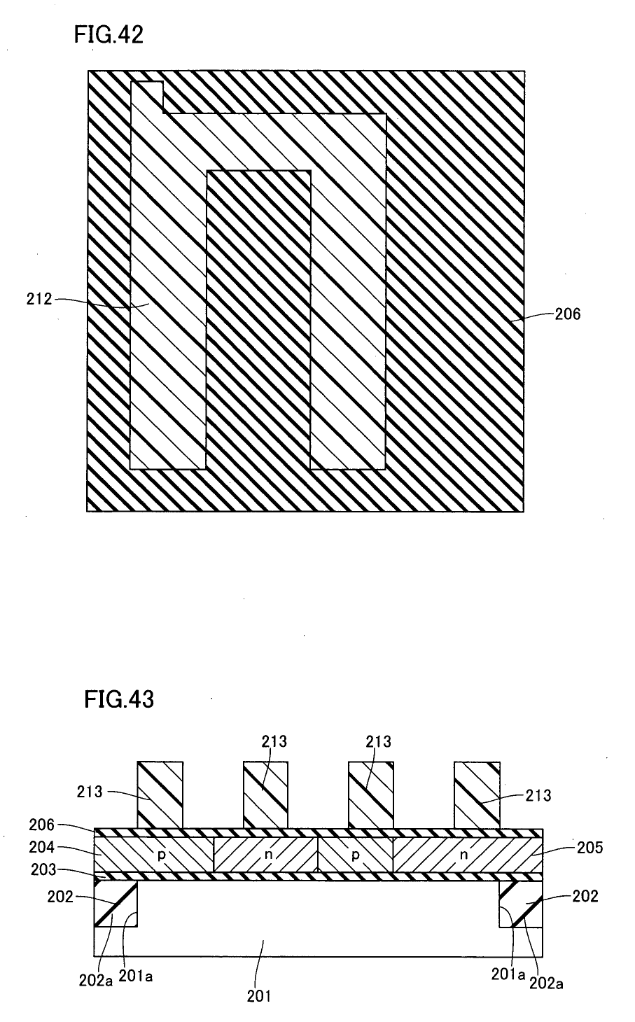

As shown in FIGS. 41 and 42, a U-shaped resist film 212 is formed in plan view. Then the resist film 212 is employed as a mask for ion-implanting phosphorus (P) into the p-type polysilicon layer 204 under a condition of implantation energy of about 50 keV and a dose (implantation dosage) of about 3×10⁻¹⁵ cm⁻² to about 5×10⁻¹⁵ cm⁻². Thus, the p-type and n-type polysilicon layers 204 and 205 having U-shapes in plan view are formed so as to be in contact with each other. Thereafter the resist film 212 is removed.

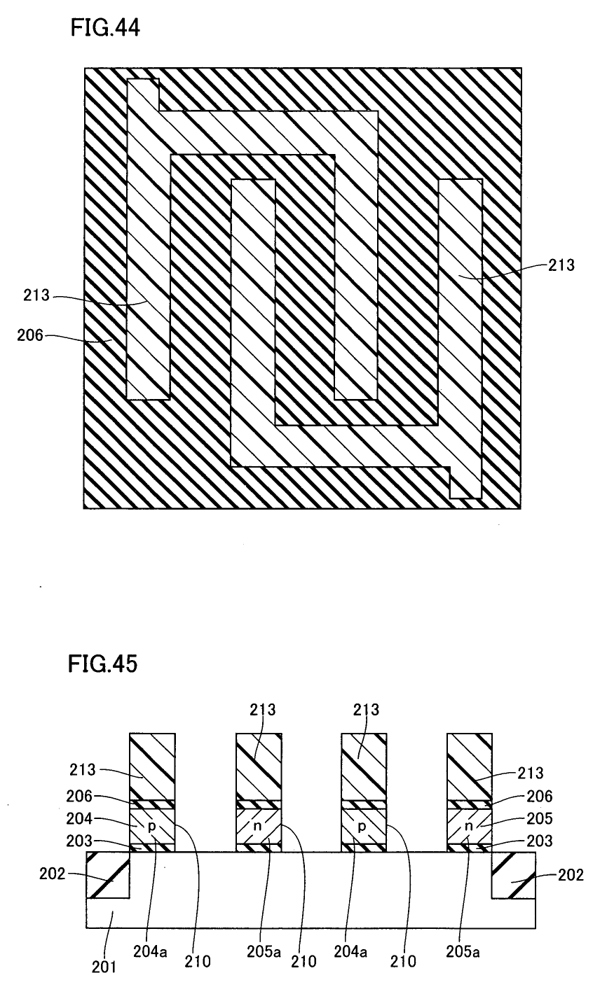

As shown in FIGS. 43 and 44, resist films 213 are formed by photolithography so as to cover the regions where the p-type and n-type polysilicon layers 204 and 205 shown in FIGS. 29 and 30 are formed. Thereafter the resist films 213 are employed as masks for patterning the insulating layers 203, the p-type polysilicon layer 204, the n-type polysilicon layer 205 and the insulating layer 206 by etching. Thus, the U-shaped two electrode sections 204a of the p-type polysilicon layer 204 and the U-shaped two electrode sections 205a of the n-type polysilicon layer 205 are formed at the horizontal intervals D (see FIG. 31) each of about 0.1 μm to about 1.0 μm as shown in FIG. 45. In other words, the three grooves 210 each having a horizontal width of about 0.1 μm to about 1.0 μm are provided between the electrode sections 204a of the p-type polysilicon layer 204 and the electrode sections 205a of the n-type polysilicon layer 205. Thereafter the resist films 213 are removed.

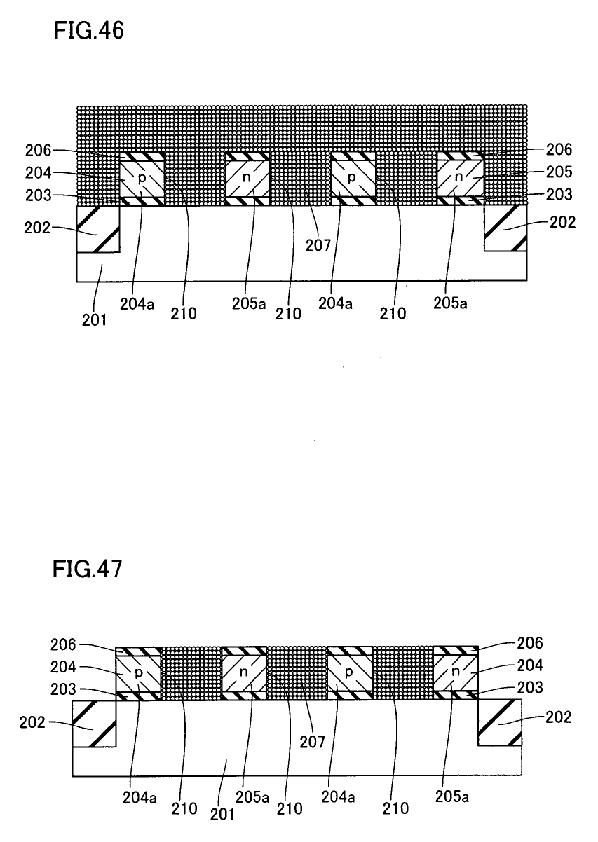

As shown in FIG. 46, silicon nanoparticles each having a particle size of about 1 nm are so deposited as to be embedded in the grooves 210 with about 100 nm to about 300 nm by cluster beam method. The cluster beam method is a method in which cluster particles are generated by flocculating Si vaporized by applying a laser beam to a solid sample made of Si in inert gas such as helium gas and evaporating the cluster particles on an objective sample. At this time, the vaporized Si and shock wave generated in the helium gas are collided, whereby Si vapor stops at a prescribed position in the helium gas. Thus, the Si vapor grows into cluster particles under given conditions and hence cluster particles homogeneous in size and inner structure are generated.

Then the silicon nanoparticles deposited on the p-type and n-type polysilicon layers 204 and 205 are removed by CMP and are flattened such that the upper surfaces of the silicon nanoparticle layers 207 and the upper surfaces of the insulating layers 206 on the p-type and n-type polysilicon layers 204 and 205 are aligned with each other. Thereafter portions where the unnecessary silicon nanoparticles are deposited are removed by photolithography and etching. Thus, the silicon nanoparticle layers 207 made of the silicon nanoparticles are formed on the grooves 210 between the n-type polysilicon layer 205 and the p-type polysilicon layer 204, thereby brining into a state shown in FIG. 47. After forming the contact holes 206a and 206b (see FIGS. 32 and 33) on the insulating layers 206 by photolithography and etching, the voltage supply electrodes 208 and 209 made of Al are so formed as to be connected to the p-type and n-type polysilicon layers 204 and 205 through the contact holes 206a and 206b respectively. Thus, the ultraviolet radiation sensor 200 according to the fifth embodiment shown in FIG. 29 is formed.

According to the fifth embodiment, as hereinabove described, the p-type and n-type polysilicon layers 204 and 205 arranged at the horizontal intervals each of about 0.1 μm to about 1.0 μm and the silicon nanoparticle layers 207 made of the silicon nanoparticles so arranged as to be embedded in the grooves 210 between the p-type polysilicon layer 204 and the n-type polysilicon layer 205 are provided on the silicon substrate 201, whereby the p-type and n-type polysilicon layers 204 and 205 are horizontally arranged and hence no electrode absorbing the ultraviolet radiation may be arranged on the light-receiving surface (upper surface) receiving the ultraviolet radiation of the silicon nanoparticle layers 207. Thus, the silicon nanoparticle layers 207 can directly receive the ultraviolet radiation. Thus, all the ultraviolet radiation incident from the light-receiving surface of the silicon nanoparticle layers 207 can be received and hence the photosensitivity of the ultraviolet radiation can be increased.

According to the fifth embodiment, as hereinabove described, the two electrode sections 204a of the p-type polysilicon layer 204 and the two electrode sections 205a of the n-type polysilicon layer 205 are so arranged as to be opposed to each other at the horizontal intervals each of about 0.1 μm to about 1.0 μm, whereby the three grooves 210 are formed between the electrode sections 204a of the p-type polysilicon layer 204 and the electrode sections 205a of the n-type polysilicon layer 205, and hence the area of the surfaces receiving the ultraviolet radiation of the silicon nanoparticle layers 207 arranged on the three grooves 210 can be increased. Consequently, the amount of the ultraviolet radiation received by the silicon nanoparticle layers 207 is increased and hence the photosensitivity of the ultraviolet radiation can be increased.

According to the fifth embodiment, as hereinabove described, the silicon nanoparticle layers 207 made of the silicon nanoparticles having a particle size (about 1 nm) capable of having a band gap of at least about 3.1 eV is employed, whereby electrons can be excited from the silicon nanoparticles with the ultraviolet radiation having a wavelength of at most about 400 nm (energy of at least about 3.1 eV) while inhibiting electrons from being excited from the silicon nanoparticles with the visible light having the wavelength longer than about 400 nm (energy of less than about 3.1 eV). Consequently, electrons can be excited from the silicon nanoparticles over a band gap of at least about 3.1 eV only when receiving the ultraviolet radiation having the wavelength of at most about 400 nm, and hence the ultraviolet radiation sensor detecting only the ultraviolet radiation can be easily formed.

According to the fifth embodiment, as hereinabove described, the insulating layers 203 of SiO₂ is provided between the silicon substrate 201 and the p-type and n-type polysilicon layers 204 and 205, whereby electrical connection between the p-type and n-type polysilicon layers 204 and 205 and the silicon substrate 201 can be suppressed by the insulating layers 203 between the p-type and n-type polysilicon layers 204 and 205 and the silicon substrate 201 also when the p-type and n-type polysilicon layers 204 and 205 are formed on the upper side of the silicon substrate 201. Consequently, a voltage is applied between the p-type polysilicon layer 204 and the n-type polysilicon layer 205, whereby the electrons excited from the silicon nanoparticles of the silicon nanoparticle layers 207 can be easily detected as a current flowing between the p-type polysilicon layer 204 and the n-type polysilicon layer 205.

### Sixth Embodiment

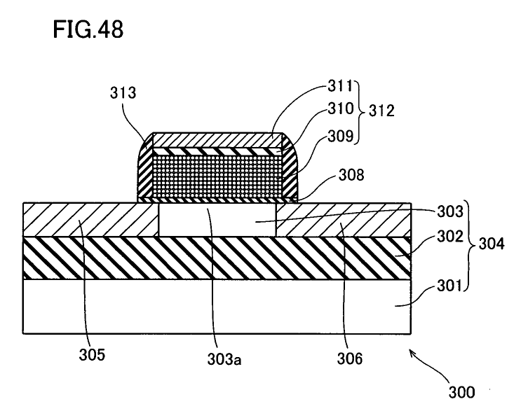

A structure of a field-effect transistor 300 according to a sixth embodiment of the present invention will be now described with reference to FIG. 48.

The field-effect transistor 300 according to the sixth embodiment comprises a source region 305 and a drain region 306 in a single-crystalline silicon layer 303 on a SOI (silicon on insulator) substrate 304 formed by a p-type silicon substrate 301, a buried oxide film 302 and a single-crystalline silicon layer 303. A surface side of the single-crystalline silicon layer 303 between the source region 305 and the drain region 306 functions as a channel layer 303a. A gate insulating film 308 is formed on the single-crystalline silicon layer 303 (channel layer 303a), the source region 305 and the drain region 306. A gate electrode 312 formed by a silicon nanoparticle layer 309, a silicon oxide layer 310 and an Au electrode layer 311 is provided on the gate insulating film 308. Side wall films (side walls) 313 made of an insulating film are provided on side surface sections of the gate electrode 312. The p-type silicon substrate 301 is an example of the “semiconductor substrate” in the present invention and the silicon nanoparticle layer 309 is an example of the “light-receiving layer” in the present invention.

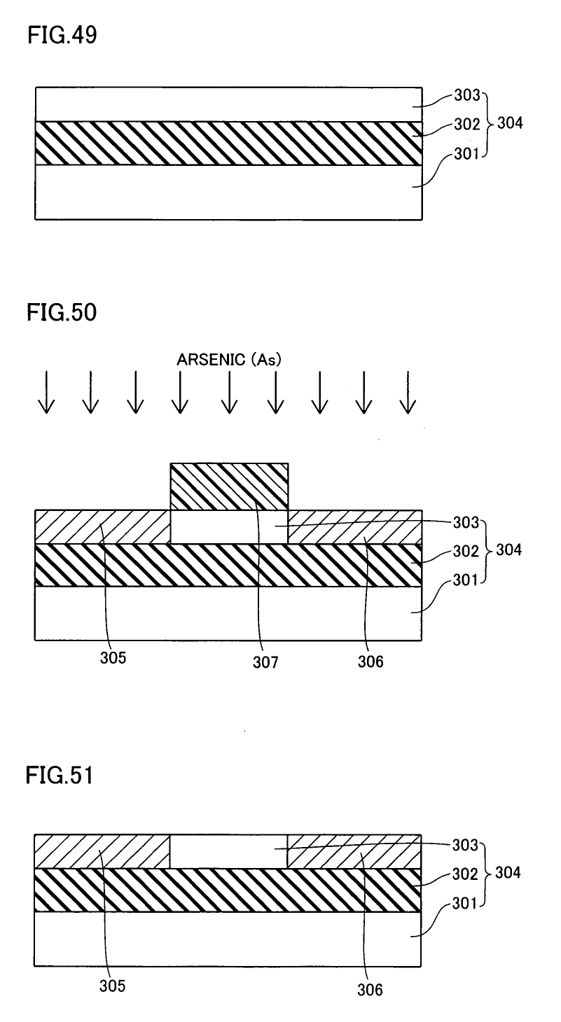

A process of fabricating the field-effect transistor 300 according to the sixth embodiment will be described with reference to FIGS. 49 to 54.

As shown in FIG. 49, the SOI substrate 304 formed by the p-type silicon substrate 301, the buried oxide film 302 and the single-crystalline silicon layer 303 is prepared. To employ the SOI substrate 304 as a substrate is because generation of carriers on the channel layer 303a (see FIG. 48) formed in the single-crystalline silicon layer 303 with visible light is prevented. The thickness of the single-crystalline silicon layer 303 is 10 to 200 nm and more preferably 50 nm. The thickness of the buried oxide film 302 is 50 to 200 nm and more preferably 100 nm.

As shown in FIG. 50, a resist film 307 for forming the source region 305 and the drain region 306 is formed on the SOI substrate 304. Thereafter a source and drain forming impurity is implanted into the regions by ion implantation. Thus, the source region 305 and the drain region 306 are formed. Arsenic (As) is implanted under ion implantation conditions of acceleration energy of 50 keV and a dose of 5×10¹⁵ cm⁻², for example.

As shown in FIG. 51, after peeling the resist film 307, the source region 305 and the drain region 306 are activated by thermal treatment (750° C., 30 minutes, an N₂ atmosphere).

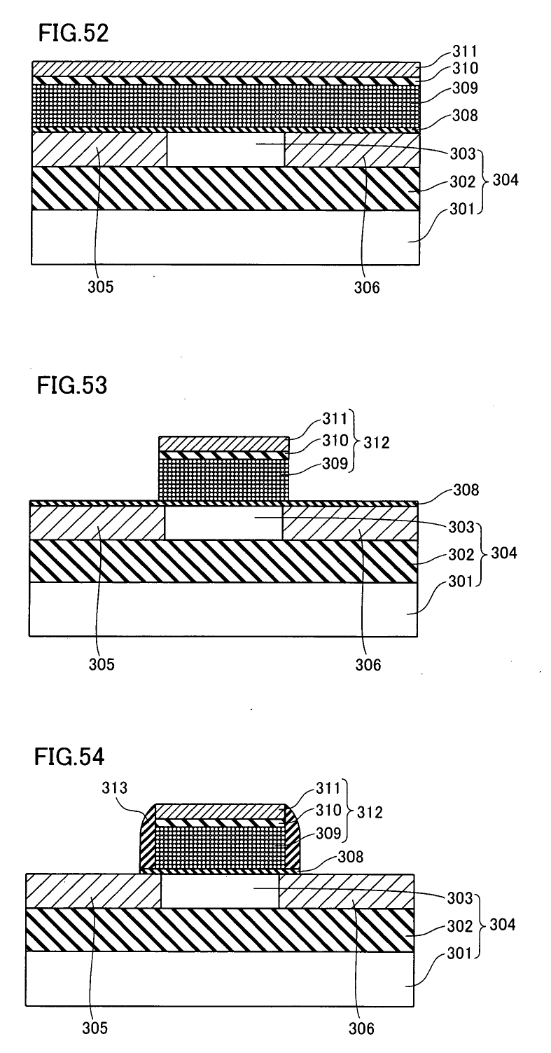

As shown in FIG. 52, the gate insulating film 308 made of a silicon oxide film is formed on the SOI substrate 304 by thermal oxidation or CVD. The thickness of the gate insulating film 308 is about 1 to about 10 nm and more preferably 2 nm. Thereafter the silicon nanoparticle layer 309 is deposited by cluster beam method and the silicon oxide layer 310 and a gold (Au) electrode layer 311 are formed on the silicon nanoparticle layer 309 by CVD and sputtering.

In the silicon nanoparticle layer 309, the particle size of each silicon nanoparticle is about 1 nm and the deposition thickness thereof is 100 nm. The silicon nanoparticle layer 309 is formed by generating silicon atom vapor with application of laser to a silicon (Si) solid sample, introducing the vapor into helium gas, forming the silicon nanoparticles with application of shock wave to the helium gas and depositing the same on the substrate, for example.

The thicknesses of the silicon oxide layer 310 and the Au electrode layer 311 are about 5 nm. While the Au electrode layer 311 is formed on the silicon oxide layer 310, an ITO (indium tin oxide) film may be employed for example so far as it is a conductive material through which ultraviolet radiation is transmitted.

The particle size of the silicon nanoparticle layer 309 is preferably at least 0.4 nm and not more than 2 nm. Thus, the band gap of the silicon nanoparticle layer 309 is expand to at least 3.0 eV, and electrons are not excited from a valence band to a conduction band with visible light having a wavelength longer than 400 nm, and electrons are selectively excited only with ultraviolet radiation having a wavelength of at most 400 nm.

When the particle size is less than 0.4 nm, a silicon particle layer is one silicon atom, which can not form a nanoparticle layer and does not function as a light-receiving layer. When the particle size is more than 2 nm, on the other hand, the band gap is less than 3 eV and electrons are excited also with light other than the ultraviolet radiation.

As shown in FIG. 53, unnecessary portions of the Au electrode layer 311, the silicon oxide layer 310 and the silicon nanoparticle layer 309 are removed by general photolithography and etching. Thus, the desired patterned gate electrode 312 is formed.

As shown in FIG. 54, the silicon oxide film is formed by CVD and then the overall surface thereof are etched baked by dry etching, thereby forming the side wall films 313 made of the silicon oxide film called the side walls on the side surface sections of the gate electrode 312. For example, the silicon oxide film is formed by thermally treating a gas mixture of tetraethoxysilane (TEOS)/oxygen (O₂) at about 720° C., and the thickness thereof is about 10 nm to about 200 nm and more preferably about 100 nm.

The field-effect transistor 300 according to the sixth embodiment of the present invention as shown in FIG. 48 is fabricated through the aforementioned steps.

A specific detecting principle of ultraviolet radiation will be hereafter described.

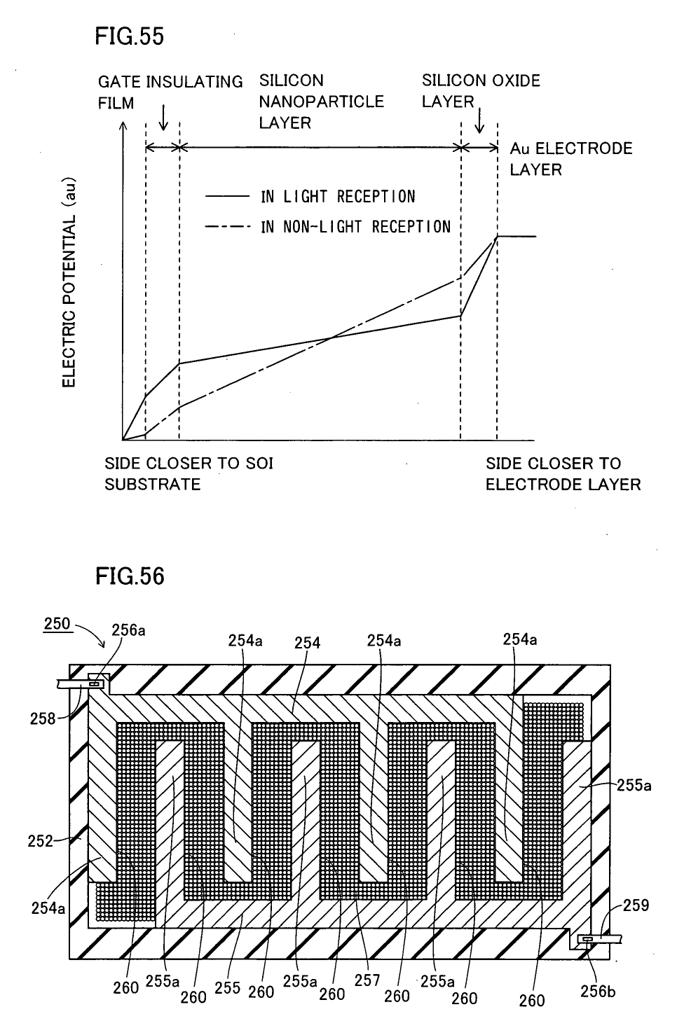

In the aforementioned field-effect transistor 300, light is incident from a side closer to the Au electrode layer 311 transparent with respect to the ultraviolet radiation having a wavelength of at most 400 nm, thereby generating electrons and holes on the silicon nanoparticle layer 309. At this time, a voltage is applied to the Au electrode layer 311, whereby electrons move to the side closer to the Au electrode layer 311 to be stored in an interface between the silicon nanoparticle layer 309 and the silicon oxide layer 310 and holes moves to a side closer to the SOI substrate 304 to be stored in an interface between the silicon nanoparticle layer 309 and the gate insulating film 308. As shown in FIG. 55, the electric potential of the gate electrode 312 in a state where the ultraviolet radiation is not incident (in non-light reception) slowly reduces from the side closer to the Au electrode layer 311 toward the side closer to the SOI substrate 304 as shown by a broken line. In light reception of the ultraviolet radiation, on the other hand, the electrons are stored in the side closer to the Au electrode layer 311 and the holes are stored in the side closer to the SOI substrate 304, whereby an inner potential is generated in the silicon nanoparticle layer 309 and the electric potential is changed to a state shown by a solid line. The potential of the holes stored in the side closer to the SOI substrate 304 increases, whereby the channel layer (inversion layer) 303a of the electrons is formed between the single-crystalline silicon layer 303 and the gate insulating film 308. At this time, if voltages (source region 305: 0 V, drain region 306: 1 V, for example) are previously applied to the source region 305 and the drain region 306 arranged on both end sides of the channel layer (inversion layer) 303a of the electrons, a current flows between the source region 305 and the drain region 306 by a voltage to applied the gate electrode 312. The current flowing between the source region 305 and the drain region 306 depends on the voltages applied to the source region 305 and the drain region 306. Therefore the current obtained by incidence of the ultraviolet radiation (current flowing between the source region 305 and the drain region 306) can be set such that a gain is large with respect to the amount of incident light and hence ultraviolet radiation can be detected with high photosensitivity.

According to this structure, the ultraviolet radiation is transmitted through the Au electrode layer 311 and the silicon oxide layer 310 and then incident. Light is incident upon the silicon nanoparticle layer 309 through the silicon oxide layer 310 and the Au electrode layer 311 transparent with respect to the ultraviolet radiation and hence the absorbed amount of light in transmission is reduced as compared with a case of incidence through a conventional n-type amorphous silicon layer. Consequently, the amount of light reaching the silicon nanoparticle layer 309 is increased and detection photosensitivity of the ultraviolet radiation can be improved as compared with a conventional case.

In the field-effect transistor 300 of the present invention, an equal number of the electrons and the holes remain in the vicinities of the interface between the silicon nanoparticle layer 309 and the gate insulating film 308 and the interface between the silicon nanoparticle layer 309 and the silicon oxide layer 310 in the silicon nanoparticle layer 309 after detecting the ultraviolet radiation. In order to cause these carriers (electrons and holes) to disappear, when the voltage applied to the Au electrode layer 311 is set to 0 V or a negative voltage, the carriers diffuse and collide with each other and disappear, whereby the field-effect transistor 300 can detect the ultraviolet radiation again.

According to the sixth embodiment, as hereinabove described, the ultraviolet radiation having a wavelength of at most 400 nm can be selectively detected with the silicon nanoparticle layer 309, pairs of the electrons and the holes generated with the ultraviolet radiation incident upon the silicon nanoparticle layer 309 are amplified, and hence the ultraviolet radiation can be detected with high photosensitivity. Light is incident upon the silicon nanoparticle layer 309 through the silicon oxide layer 310 and the Au electrode layer 311 transparent with respect to the ultraviolet radiation and hence the absorbed amount of the light in transmission is reduced as compared with the case of incidence through the conventional n-type amorphous silicon layer and reduction in the detection photosensitivity of the ultraviolet radiation can be suppressed.

The embodiments disclosed this time must be considered as illustrative and not restrictive in all points. The range of the present invention is shown not by the above description of the embodiments but by the scope of claim for patent, and all modifications within the meaning and range equivalent to the scope of claim for patent are included.

For example, while the two-dimensional CCD is employed as the image detecting portion in each of the aforementioned first to fourth embodiments, the present invention is not restricted to this but the ultraviolet radiation sensor according to the fifth embodiment or the field-effect transistor according to the sixth embodiment may be employed as the image detecting portion.

While the present invention is applied to the cellular phone, the personal digital assistant, the laptop personal computer and the digital camera has been shown in each of the aforementioned first and second embodiments, the present invention is not restricted to this but is also applicable to an information terminal other than the cellular phone, the personal digital assistant, the laptop personal computer and the digital camera (electronic still camera). As the information terminal other than the cellular phone, the personal digital assistant, the laptop personal computer and the digital camera (electronic still camera) includes a portable audio player and a watch, for example.

While the ultraviolet LED is provided on the information terminal in each of the aforementioned first and second embodiments, the present invention is not restricted to this but no ultraviolet LED may be provided on the information terminal.

While the information terminal has either a function of confirming the portion where the pigmented spot on the skin of the human body exists or a function of distinguishing between the high maturity vegetable and the low maturity vegetable in each of the aforementioned first and second embodiments, the present invention is not restricted to this but the information terminal may be so formed as to have both the functions of confirming the portion where the pigmented spot on the skin of the human body exists and distinguishing between the high maturity vegetable and the low maturity vegetable.

While the image of the human body or the vegetable by the ultraviolet radiation is displayed on the liquid crystal display in each of the aforementioned first to third embodiments, the present invention is not restricted to this but ultraviolet radiation information such as the amount or intensity of ultraviolet radiation may be displayed on the liquid crystal display in addition to the image of the human body or the vegetable by the ultraviolet radiation. According to this structure, the ultraviolet radiation information such as the amount or intensity of ultraviolet radiation can be reliably grasped, and hence implementation of a measure for the ultraviolet radiation on the basis of the ultraviolet radiation information from the information terminal can inhibit the immunity of the object from disadvantageous reduction due to ultraviolet radiation, according to each of the first and second embodiments.

While a case of distinguishing the maturity of the vegetable are described in each of the aforementioned second and third embodiments, the present invention is not restricted to this but the maturity of food other than the vegetables can be also distinguished so far as the food contains the antioxidant substance absorbing the ultraviolet radiation. The food containing the antioxidant substance absorbing the ultraviolet radiation other than the vegetables includes fruits and rice, for example.

While both of the image of the vegetable by the ultraviolet radiation and the bar graph or the indicator showing the maturity of the vegetable are displayed in each of the aforementioned second and third embodiments, the present invention is not restricted to this but only the image of the vegetable by the ultraviolet radiation may be displayed or only the bar graph or the indicator showing the maturity of the vegetable may be displayed.

While the display color of the vegetable containing the large quantity of antioxidant substances is deeper than that of the vegetable containing the small quantity of antioxidant substances in the aforementioned third embodiment, the present invention is not restricted to this but the vegetable containing the large quantity of antioxidant substances and the vegetable containing the small quantity of antioxidant substances are displayed in different display colors respectively.

While the pollen is shown as the substance absorbing the ultraviolet radiation in the aforementioned fourth embodiment, the present invention is not restricted to this but a fabric having a large ultraviolet radiation absorptance as a measure for the ultraviolet radiation may be employed as the substance absorbing the ultraviolet radiation.

While the silicon nanoparticle layers are embedded between the electrode sections of the n-type polysilicon layer and the electrode sections of the p-type polysilicon layer in the aforementioned fifth embodiment, the present invention is not restricted to this but a semiconductor layer other than the silicon nanoparticle layer such as diamond may be employed as the semiconductor layer capable of detecting the ultraviolet radiation.

While the electrode sections of the n-type polysilicon layer and the electrode sections of the p-type polysilicon layer are arranged at the horizontal prescribed intervals in the aforementioned fifth embodiment, the present invention is not restricted to this but the electrode sections of the n-type polysilicon layer and the electrode sections of the p-type polysilicon layer may be arranged at prescribed intervals along the surface of the silicon substrate so as not to cover the light-receiving surfaces (upper surface) side of the silicon nanoparticle layers between the electrode sections of the n-type polysilicon layer and the electrode sections of the p-type polysilicon layer.

While silicon nanoparticle layers made of the silicon nanoparticles are provided between the n-type polysilicon layer and the p-type polysilicon layer different in a polarity in the aforementioned fifth embodiment, the present invention is not restricted to this but the silicon nanoparticle layers may be provided between the n-type polysilicon layers identical in the polarity, or the silicon nanoparticle layers may be provided between the p-type polysilicon layers.

While the silicon nanoparticle layers are formed in the grooves between the p-type polysilicon layer and n-type polysilicon layer formed in the U-shapes in plan view in the aforementioned fifth embodiment, the present invention is not restricted to this but a silicon nanoparticle layers 257 may be provided in seven grooves 260 between a comb-shaped p-type polysilicon layer 254 having a plurality of electrode section 254a (four in a modification in FIG. 56) and a comb-shaped n-type polysilicon layer 255 having a plurality of electrode section 255a (four in a modification in FIG. 56) as in an ultraviolet radiation sensor 250 according to the modification shown in FIG. 56. In this case, the area of receiving the ultraviolet radiation of silicon nanoparticle layer 257 is increased, and hence the amount of receiving the ultraviolet radiation can be increased. Consequently, photosensitivity of the ultraviolet radiation can be further improved. According to the modification shown in FIG. 56, an element isolation region 252 formed so as to surrounding an element forming region, a contact hole 256a for connecting an after-mentioned voltage supply electrode 258 to the p-type polysilicon layer 254, a contact hole 256b for connecting an after-mentioned voltage supply electrode 259 to the n-type polysilicon layer 255, a voltage supply electrode 258 for applying a voltage to the p-type polysilicon layer 254 and a voltage supply electrode 259 for applying a voltage to the n-type polysilicon layer 255 are provided similarly to the aforementioned fifth embodiment.

While the p-type polysilicon layer and the n-type polysilicon layer are employed as the electrodes in the aforementioned fifth embodiment, the present invention is not restricted to this but single-crystalline silicon or amorphous silicon other than polysilicon may be employed as the electrode. Alternatively, a semiconductor other than silicon or a metal other than semiconductor may be employed.

While the insulating layer is provided between the n-type or p-type silicon substrate and the p-type and n-type polysilicon layers in the aforementioned fifth embodiment, the present invention is not restricted to this but the p-type polysilicon layer and n-type polysilicon layer may be directly provided on the insulating substrate.

While the field-effect transistor is formed by employing the SOI substrate in the aforementioned sixth embodiment, the present invention is not restricted to this but the field-effect transistor may be formed on the single-crystalline silicon substrate generally employed.

1-2. (canceled)

3. An electric device comprising:
image detecting portion (6, 66, 127, 149) for receiving ultraviolet radiation and detecting an image by received said ultraviolet radiation; and
a display section (2, 32, 42, 52, 62, 82, 92, 102, 126, 147, 172) for displaying ultraviolet radiation information generated on the basis of said image by said ultraviolet radiation detected with said image detecting portion, wherein
said image detecting portion includes a field-effect transistor having a semiconductor substrate, source and drain regions provided on said semiconductor substrate, a channel layer formed between said source and drain regions, a gate insulating film formed on said channel layer, and a gate electrode formed on said gate insulating film and formed with a light-receiving layer receiving said ultraviolet radiation to generate electrons and holes, a silicon oxide layer and an electrode layer in an order from a side closer to said gate insulating film.

4-27. (canceled)

28. A field-effect transistor comprising:
a semiconductor substrate (301);
a source region (305) and a drain region (306) provided on said semiconductor substrate;
a channel layer (303a) formed between said source and drain regions; and
a gate insulating film (308) formed on said channel layer and a gate electrode (312) formed on said gate insulating film, wherein
said gate electrode includes a light-receiving layer (309) receiving ultraviolet radiation to generate electrons and holes, a silicon oxide layer (310) and an electrode layer (311) in an order from a side closer to said gate insulating film.

29. The field-effect transistor according to claim 28, wherein a particle size of each silicon nanoparticle of said light-receiving layer is at least 0.4 nm and not more than 2 nm.
